(19) United States
(12) Patent Application Publication
(10) Pub. No.: US 2011/0053918 A1
(43) Pub. Date: Mar. 3, 2011

(54) GAMMA SECRETASE MODULATORS

(76) Inventors: Zhaoning Zhu, Plainsboro, NJ (US); William J. Greenlee, Teaneck, NJ (US); Robert D. Mazzola, JR., Stewartsville, NJ (US); Jun Qin, Edison, NJ (US); Xianhai Huang, Warren, NJ (US); Anandan Palani, Bridgewater, NJ (US)

(21) Appl. No.: 12/665,551

(22) PCT Filed: Jun. 27, 2008
(86) PCT No.: PCT/US08/08042

§ 371 Date: Oct. 26, 2010

Related U.S. Application Data
(60) Provisional application No. 60/947,176, filed on Jun. 29, 2007.

(51) Int. Cl. A61K 31/541; C07D 239/70; C07D 239/24; C07D 233/56; C07D 403/12; C07D 401/10; C07D 279/02; C07D 413/10; C07D 265/36; C07D 241/36; A61K 31/4353; A61K 31/519; A61K 31/505; A61K 31/4166; A61K 31/4439; A61K 31/5355; A61K 31/538; A61K 31/497; A61P 25/28; A61P 9/10; A61P 25/00

(52) U.S. Cl. 514/222.2; 544/282; 544/317; 548/315.4; 548/313.7; 546/275.1; 544/3; 544/96; 544/105; 544/349; 514/319; 514/259.1; 514/256; 514/389; 514/341; 514/230.5; 514/228.8; 514/252.1

(57) ABSTRACT

In its many embodiments, the present invention provides a novel class of heterocyclic compounds as modulators of gamma secretase, methods of preparing such compounds, pharmaceutical compositions containing one or more such compounds, methods of preparing pharmaceutical formulations comprising one or more such compounds, and methods of treatment, prevention, inhibition, or amelioration of one or more diseases associated with the central nervous system using such compounds or pharmaceutical compositions.

### REFERENCE TO RELATED APPLICATION

This application claims the benefit of U.S. Provisional Application Ser. No. 60/947,176 filed Jun. 29, 2007.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the amyloid protein is critical for the seeding of amyloid formation: implications for the pathogenesis of Alzheimer's disease, Biochemistry, May 11, 1993, 32(18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249).

Furthermore, it is known that mutations of APP and presenelin genes, which is observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272(51), p. 32247-32253). Therefore, compounds which reduce production of Aβ40 and Aβ42 are expected as an agent for controlling progress of Alzheimer's disease or for preventing the disease.

These Aβs are produced when APP is cleaved by beta secretase and subsequently clipped by gamma secretase. In consideration of this, creation of inhibitors of γ secretase and β secretase has been attempted for the purpose of reducing production of Aβs. Many of these secretase inhibitors already known are peptides or peptidomimetics such as L-685,458. L-685,458, an aspartyl protease transition stale mimic, is a potent inhibitor of amyloid β-protein precursor γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

The compounds of this invention (Formula I) can be useful as gamma secretase modulators and can be useful in the treatment and prevention of diseases such as, for example, Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), Olfactory function loss (Getchell, et. al. Neurobiology of Aging, 663-673, 24, 2003).

In one embodiment, the present application discloses a compound, or pharmaceutically acceptable salts, solvates, esters or prodrugs of said compound, said compound having the general structure shown in Formula I:

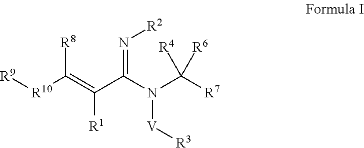

wherein R¹, R², R³, R⁴, R⁶, R⁷, R⁸, R⁹, R¹⁰, and V are as defined below.

In another embodiment, the present application discloses a compound, or pharmaceutically acceptable salts, solvates, esters or prodrugs of said compound, said compound having the general structure shown in Formula IA:

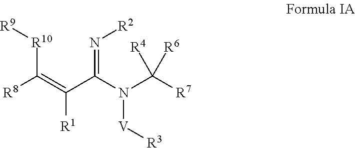

wherein R¹, R², R³, R⁴, R⁶, R⁷, R⁸, R⁹, R¹⁰, and V are as defined below.

The compounds of Formula I can be useful as gamma secretase modulators and can be useful in the treatment and prevention of diseases such as, for example, central nervous system disorders such as Alzheimers disease and Downs Syndrome.

This invention also provides compounds of formula I.

This invention also provides pharmaceutically acceptable salts of the compounds of formula I.

This invention also provides pharmaceutically acceptable esters of the compounds of formula I.

This invention also provides pharmaceutically solvates of the compounds of formula I.

This invention also provides compounds of formula I in pure and isolated form.

This invention also provides compounds of formula I in pure form.

This invention also provides compounds of formula I in isolated form.

This invention also provides compounds 1 to 48.

This invention also provides pharmaceutical compositions comprising an effective amount of one or more (e.g., one) compounds of formula I, or a pharmaceutically acceptable salt, ester or solvate thereof, and a pharmaceutically acceptable carrier.

This invention also provides pharmaceutical compositions comprising an effective amount of one or more (e.g., one) compounds of formula I, or a pharmaceutically acceptable salt, ester or solvate thereof, and an effective amount of one or more (e.g., one) other pharmaceutically active ingredients (e.g., drugs), and a pharmaceutically acceptable carrier.

This invention also provides a method for modulating (including inhibiting, antagonizing and the like) gamma-secretase, comprising administering an effective (i.e., therapeutically effective) amount of one or more compounds of formula I to a patient in need of treatment.

This invention also provides a method for modulating (including inhibiting, antagonizing and the like) gamma-secretase, comprising administering an effective (i.e., therapeutically effective) amount of a compound of formula I to a patient in need of treatment.

This invention also provides a method of treating one or more neurodegenerative diseases, comprising administering an effective (i.e., therapeutically effective) amount of one or more compounds of formula I to a patient in need of treatment.

This invention also provides a method of treating one or more neurodegenerative diseases, comprising administering an effective (i.e., therapeutically effective) amount of a compound of formula I to a patient in need of treatment.

This invention also provides a method of inhibiting the deposition of amyloid protein (e.g., amyloid beta protein) in, on or around neurological tissue (e.g., the brain), comprising administering an effective (i.e., therapeutically effective) amount of one or more compounds of formula I to a patient in need of treatment.

This invention also provides a method of inhibiting the deposition of amyloid protein (e.g., amyloid beta protein) in, on or around neurological tissue (e.g., the brain), comprising administering an effective (i.e., therapeutically effective) amount of a compound of formula I to a patient in need of treatment.

This invention also provides a method of treating Alzheimer's disease, comprising administering an effective (i.e., therapeutically effective) amount of one or more compounds of formula I to a patient in need of treatment.

This invention also provides a method of treating Alzheimer's disease, comprising administering an effective (i.e., therapeutically effective) amount of a compound of formula I to a patient in need of treatment.

This invention also provides a method of treating Alzheimer's disease, comprising administering an effective (i.e., therapeutically effective) amount of one or more compounds of formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Alzheimer's disease, comprising administering an effective (i.e., therapeutically effective) amount of one or more compounds of formula I, in combination with an effective (i.e., therapeutically effective) amount of one or more compounds selected from the group consisting of Aβ antibody inhibitors, gamma secretase inhibitors and beta secretase inhibitors.

This invention also provides combinations comprising an effective (i.e., therapeutically effective) amount of one or more compounds of formula I, in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

This invention also provides a method of treating Alzheimer's disease, comprising administering an effective (i.e., therapeutically effective) amount of a compound of formula I, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of one or more compounds of formula I to a patient in need of treatment.

This invention also provides a method of treating Downs syndrome, comprising administering an effective (i.e., therapeutically effective) amount of a compound of formula I to a patient in need of treatment.

This invention also provides a method of treating Downs syndrome, comprising administering an effective (i.e., therapeutically effective) amount of one or more compounds of formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of a compound of formula I, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula I and the administration of an effective amount of one or more (e.g., one) other pharmaceutical active ingredients (e.g., drugs).

This invention also provides a method of treating mild cognitive impairment, comprising administering an effective amount of one or more (e.g., one) compounds of formula I to a patient in need of treatment.

This invention also provides a method of treating glaucoma, comprising administering an effective amount of one or more (e.g., one) compounds of formula I to a patient in need of treatment.

This invention also provides a method of treating cerebral amyloid angiopathy, comprising administering an effective amount of one or more (e.g., one) compounds of formula I to a patient in need of treatment.

This invention also provides a method of treating stroke, comprising administering an effective amount of one or more (e.g., one) compounds of formula I to a patient in need of treatment.

This invention also provides a method of treating dementia, comprising administering an effective amount of one or more (e.g., one) compounds of formula I to a patient in need of treatment.

This invention also provides a method of treating microgliosis, comprising administering an effective amount of one or more (e.g., one) compounds of formula I to a patient in need of treatment.

This invention also provides a method of treating brain inflammation, comprising administering an effective amount of one or more (e.g., one) compounds of formula I to a patient in need of treatment.

This invention also provides a method of treating olfactory function loss, comprising administering an effective amount of one or more (e.g., one) compounds of formula I to a patient in need of treatment.

This invention also provides pharmaceutical compositions comprising a combination of an effective amount of one or more (e.g., one) compounds of formula I, in combination with an effective amount of one or more compounds selected from the group consisting of cholinesterase inhibitors, Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of a compound of formula I in a pharmaceutically acceptable carrier, and another container (i.e., a second container) comprises an effective amount of another pharmaceutically active ingredient, the combined quantities of the compound of formula I and the other pharmaceutically active ingredient being effective to: (a) treat Alzheimer's disease, or (b) inhibit the deposition of amyloid protein in, on or around neurological tissue (e.g., the brain), or (c) treat neurodegenerative diseases, or (d) modulate the activity of gamma-secretase.

This invention also provides a kit comprising, in separate containers, in a single package, pharmaceutical compositions for use in combination, wherein one container comprises an effective amount of a compound of formula I in a pharmaceutically acceptable carrier, and another container (i.e., a second container) comprises an effective amount of another pharmaceutically active ingredient (as described below), the combined quantities of the compound of formula I and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

This invention also provides any one of the methods disclosed above and below wherein the compound is selected from the group consisting of the compounds 1 to 48.

Other embodiments of this invention are directed to any one of the embodiments above or below that are directed to formula I, or the use of formula I (e.g. the embodiments directed to methods of treatment, pharmaceutical compositions and kits), wherein the compound is a compound of formula IA instead of formula I. Those skilled in the art will appreciate that the compounds of formula I and formula IA are isomers of each other.

This invention also provides any one of the above mentioned methods of treatment wherein the compound of formula I is selected from the group consisting of compounds 1-48.

This invention also provides any one of the above mentioned methods of treatment wherein the compound of formula I is selected from the group consisting of the compounds in Table 1.

### DETAILED DESCRIPTION

In one embodiment, the present invention discloses compounds which are represented by structural Formula I, or a pharmaceutically acceptable salt, solvate, ester or prodrug thereof, wherein the various moieties are described below.

For the compounds of formula I and IA, R¹, R², R³, R⁴, R⁶, R⁷, R⁸, R⁹, R¹⁰, and V are each independently selected.

Thus, one embodiment of the invention is directed to a compound, or pharmaceutically acceptable salts, solvates, esters or prodrugs of said compound, said compound having the general structure shown in Formula I:

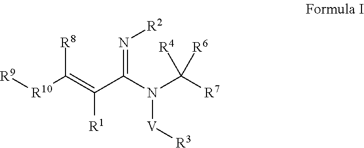

wherein:

optionally, a ring moiety is formed when either:
(i) R¹ and R³ are joined together to form:
   (a) a 5-8 membered heterocyclyl moiety that is optionally substituted with 1-5 independently selected R²¹ groups, or
   (b) a 5-8 membered heterocyclyl moiety that is optionally fused with an aryl ring or a heteroaryl ring wherein the ring moiety resulting from the fusion is optionally substituted with 1-5 independently selected R²¹ groups, or
   (c) a 5-8 membered heterocyclenyl moiety that is optionally substituted with 1-5 independently selected R²¹ groups, or
   (d) a 5-8 membered heterocyclenyl moiety that is optionally fused with an aryl ring or a heteroaryl ring wherein the ring moiety resulting from the fusion is optionally substituted with 1-5 independently selected R²¹ groups; or
(ii) R³ and R⁴ are joined together to form:
   (a) a 5-8 membered heterocyclyl moiety that is optionally substituted with 1-5 independently selected R²¹ groups, or
   (b) a 5-8 membered heterocyclyl moiety that is optionally fused with an aryl ring or a heteroaryl ring wherein the ring moiety resulting from the fusion is optionally substituted with 1-5 independently selected R²¹ groups, or
   (c) a 5-8 membered heterocyclenyl moiety that is optionally substituted with 1-5 independently selected R²¹ groups, or
   (d) a 5-8 membered heterocyclenyl moiety that is optionally fused with an aryl ring or a heteroaryl ring wherein the ring moiety resulting from the fusion is optionally substituted with 1-5 independently selected R²¹ groups; or
(iii)
   (a) R¹ and R³ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and R³ and R⁴ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, or
   (b) R¹ and R³ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and R³ and R⁴ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups;
      (i.e., the ring formed in (i), (ii) or (iii) is optional, and when a ring is formed the ring is either the ring described in (i), or the ring described in (ii) or the ring described in (iii));
      and

V is selected from the group consisting of a bond, —O—, —S(O₂)—, —S(O)—, —C(O)—, and —N(R¹⁴)—;

R¹ (when R¹ is not joined to R³), is selected from the group consisting of H, alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-5 independently selected R²¹ substituents;

or, alternatively, R¹ (when not joined with R³) and R⁸ are taken together to form a bond (i.e., there is a triple bond between the carbon atom to which R¹ was bonded to and the carbon to which R⁸ was bonded to, i.e., the compound of formula I is a compound of formula II:

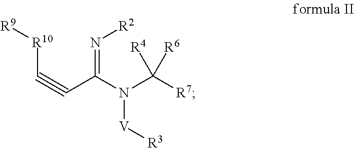

R² is independently selected from the group consisting of H, alkyl, cycloalkyl, cycloalkenyl, cycloalkylalky-, heterocycloalkyl, heterocycloalkylalkyl-, aryl, arylalkyl-, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalky-, heterocycloalkyl, heterocycloalkylalkyl-, aryl, arylalkyl-, heteroaryl, and heteroarylalkyl- is optionally substituted with 1-5 independently selected R²¹ substituents;

R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-5 independently selected R²¹ substituents;

R⁴, R⁶ and R⁷ are each independently selected from the group consisting of H, alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-5 independently selected R²¹ substituents;

R⁸ is selected from the group consisting of H, alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-3 independently selected R²¹ substituents;

R⁹ is selected from the group consisting of alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-3 independently selected R²¹ substituents,

R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl-,

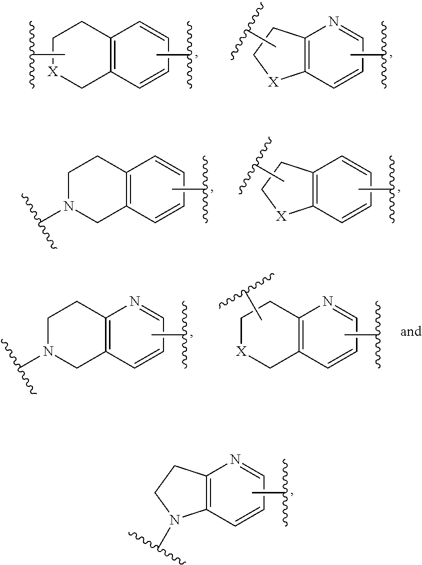

where X is O, N(R¹⁴) or S;

wherein each of said R¹⁹ groups is optionally substituted with 1-3 independently R²¹ substituents;

R¹⁴ is selected from the group consisting of H, alkyl, alkenyl, alkynyl, cycloalkyl, cycloalkenyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶); wherein each of said alkyl, alkenyl, alkynyl, cycloalkyl, cycloalkenyl, cycloalkylalkyl, cycloalkenyl, heterocyclyl, heterocyclylalkyl, aryl, arylalkyl, heteroaryl, and heteroarylalkyl is optionally substituted with 1-5 independently selected R²¹ substituents;

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ᵣ-alkyl, (R¹⁸)ᵣ-cycloalkyl, (R¹⁸)ᵣ-cycloalkylalkyl, (R¹⁸)ᵣ-heterocyclyl, (R¹⁸)ᵣ-heterocyclylalkyl, (R¹⁸)ᵣ-aryl, (R¹⁸)ᵣ-arylalkyl, (R¹⁸)ᵣ-heteroaryl and (R¹⁸)ᵣ-heteroarylalkyl, wherein each r is independently 1 to 5;

Each R¹⁸ is independently selected from the group consisting of: alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

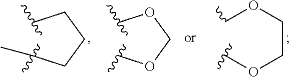

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

Each R²¹ is independently selected from the group consisting of: alkyl, alkenyl, alkynyl, cycloalkyl, cycloalkenyl, heterocycloalkyl, heterocycloalkylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁶)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ is optionally substituted by 1 to 5 independently selected R²² groups; and

Each R²² is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula I:

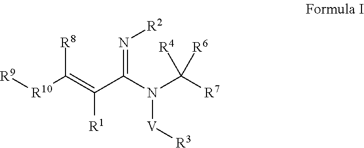

wherein:
either (i) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (ii) R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (iii) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; and R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;
   and

V is selected from the group consisting of a bond, —O—, —S(O₂)—, —S(O)—, —C(O)—, and —N(R¹⁴)—;

R¹ (when R¹ is not joined to R³), is selected from the group consisting of H, alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

or, alternatively, R¹ (when not joined with R³) and R⁸ are taken together to form a bond (i.e., there is a triple bond between the carbon atom to which R¹ was bonded to and the carbon to which R⁸ was bonded to, i.e., the compound of formula I is a compound of formula II:

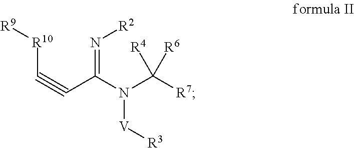

R² is independently selected from the group consisting of H, alkyl, cycloalkyl, cycloalkenyl, cycloalkylalkyl, heterocycloalkyl, heterocycloalkylalkyl, aryl, arylalkyl, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalkyl, heterocycloalkyl, heterocycloalkylalkyl-, aryl, arylalkyl-, heteroaryl, and heteroarylalkyl- can be unsubstituted or optionally independently substituted with 1-5 substituents which can be the same or different, each substituent being independently selected from the group consisting of the moieties shown below;

R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁴, R⁶ and R⁷ can be the same or different, each being independently selected from the group consisting of H, alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, with each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R¹⁴ is selected from the group consisting of H, alkyl, alkenyl, alkynyl, cycloalkyl, cycloalkenyl, cycloalkylalkyl-, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkenyl, cycloalkylalkyl-, heterocyclyl, heterocyclylalkyl-, aryl, arylalkyl-, heteroaryl, and heteroarylalkyl- can be unsubstituted or optionally independently substituted with 1-5 substituents which can be the same or different, each substituent being independently selected from the group consisting of consisting of the moieties shown below;

R⁹ is selected from the group consisting of alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

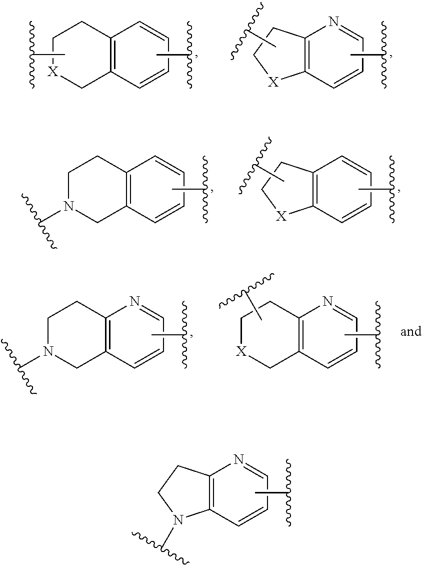

where X is O, N(R¹⁴) or S;

wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

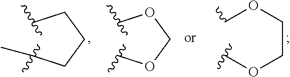

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkenyl, cycloalkylalkyl, heterocycloalkyl, heterocycloalkylalkyl, aryl, arylalkyl, heteroaryl, heteroarylalkyl, alkenyl and alkynyl groups in R¹, R², R³, R⁴, R⁶, R⁷, R⁸, R⁹, R¹⁰ and R¹⁴, or the 5-8 membered heterocyclyl or 5-8 membered heterocyclenyl moieties formed from the joining of R¹ and R³ or R³ and R⁴, are independently unsubstituted or substituted by 1 to 5 (unless indicated otherwise) R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

The statement above: “either (i) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (ii) R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (iii) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; and R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;” means that the occurrences of (i), (ii) and (iii) are mutually exclusive and that only one of (i), (ii) and (iii) can be present at any given time.

It should be understood that when R¹ and R³ are joined together to form a 5-8 membered heterocyclyl or 5-8 membered heterocyclenyl moiety, each of said heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

It should be also understood that when R³ and R⁴ are joined together to form a 5-8 membered heterocyclyl or 5-8 membered heterocyclenyl moiety, each of said heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

In another embodiment, the present application discloses a compound, or pharmaceutically acceptable salts, solvates, esters or prodrugs of said compound, said compound having the general structure shown in Formula I:

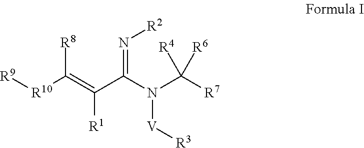

wherein:
either (i) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (ii) R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (iii) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; and R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;
   and

V is selected from the group consisting of a bond, —O—, —S(O₂)—, —S(O)—, —C(O)—, and —N(R¹⁴)—;

R¹ (when R¹ is not joined to R³), is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is independently selected from the group consisting of H, alkyl, cycloalkyl, cycloalkenyl, cycloalkylalkyl-, heterocycloalkyl, heterocycloalkylalkyl-, aryl, arylalkyl, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalkyl-, heterocycloalkyl, heterocycloalkylalkyl-, aryl, arylalkyl-, heteroaryl, and heteroarylalkyl- can be unsubstituted or optionally independently substituted with 1-5 substituents which can be the same or different, each substituent being independently selected from the group consisting of the moieties shown below;

R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁴, R⁶ and R⁷ can be the same or different, each being independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cyclaoalkenyl, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R¹⁴ is selected from the group consisting of H, alkyl, alkenyl, alkynyl, cycloalkyl, cycloalkenyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkenyl, cycloalkylalkyl, heterocyclyl, heterocyclylalkyl, aryl, arylalkyl, heteroaryl, and heteroarylalkyl can be unsubstituted or optionally independently substituted with 1-5 substituents which can be the same or different, each substituent being independently selected from the group consisting of consisting of the moieties shown below;

R⁹ is selected from the group consisting of alkyl-, alkenyl-, alkynyl-, aryl-, arylalkyl-, alkylaryl-, cycloalkyl-, cycloalkenyl, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

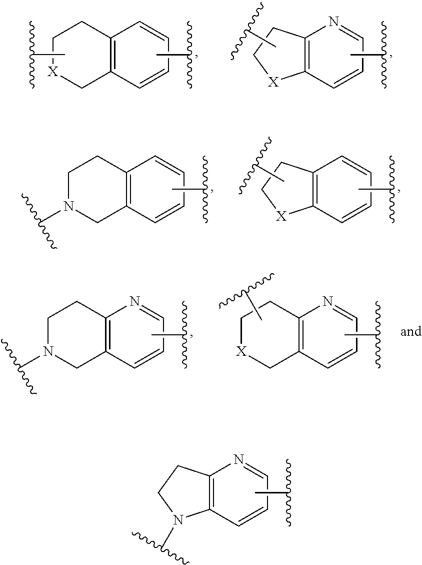

where X is O, N(R¹⁴) or S;

wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

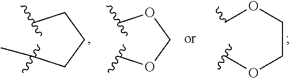

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocycloalkyl, heterocycloalkylalkyl, aryl, arylalkyl, heteroaryl, heteroarylalkyl, alkenyl and alkynyl groups in R¹, R², R³, R⁴, R⁶, R⁷, R⁸, R⁹, R¹⁰ and R¹⁴, or the 5-8 membered heterocyclyl or 5-8 membered heterocyclenyl moieties formed from the joining of R¹ and R³ or R³ and R⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment the invention is directed to a compound, or pharmaceutically acceptable salts, solvates, esters or prodrugs of said compound, said compound having the general structure shown in Formula I:

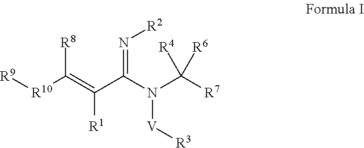

wherein:

either
(i) R¹ and R³ are joined together to form:
   (a) a 5-8 membered heterocyclyl moiety that is optionally substituted with 1-5 independently selected R²¹ groups, or
   (b) a 5-8 membered heterocyclyl moiety that is optionally fused with an aryl ring or a heteroaryl ring wherein the ring moiety resulting from the fusion is optionally substituted with 1-5 independently selected R²¹ groups, or
   (c) a 5-8 membered heterocyclenyl moiety that is optionally substituted with 1-5 independently selected R²¹ groups, or
   (d) a 5-8 membered heterocyclenyl moiety that is optionally fused with an aryl ring or a heteroaryl ring wherein the ring moiety resulting from the fusion is optionally substituted with 1-5 independently selected R²¹ groups; or
(ii) R³ and R⁴ are joined together to form:
   (a) a 5-8 membered heterocyclyl moiety that is optionally substituted with 1-5 independently selected R²¹ groups, or
   (b) a 5-8 membered heterocyclyl moiety that is optionally fused with an aryl ring or a heteroaryl ring wherein the ring moiety resulting from the fusion is optionally substituted with 1-5 independently selected R²¹ groups, or
   (c) a 5-8 membered heterocyclenyl moiety that is optionally substituted with 1-5 independently selected R²¹ groups, or
   (d) a 5-8 membered heterocyclenyl moiety that is optionally fused with an aryl ring or a heteroaryl ring wherein the ring moiety resulting from the fusion is optionally substituted with 1-5 independently selected R²¹ groups; or
(iii)
   (a) R¹ and R³ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and R³ and R⁴ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, or
   (b) R¹ and R³ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and R³ and R⁴ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups;
      and

V is selected from the group consisting of a bond, —O—, —S(O₂)—, —S(O)—, —C(O)—, and —N(R¹⁴)—;

R¹ (when R¹ is not joined to R³), is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-5 independently selected R²¹ substituents;

or, alternatively, R¹ (when not joined with R³) and R⁸ are taken together to form a bond (i.e., there is a triple bond between the carbon atom to which R¹ was bonded to and the carbon to which R⁸ was bonded to, i.e., the compound of formula I is a compound of formula II:

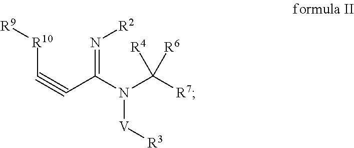

R² is independently selected from the group consisting of H, alkyl, cycloalkenyl, cycloalkylalkyl, heterocycloalkyl, heterocycloalkylalkyl-, aryl, arylalkyl-, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalkyl, heterocycloalkyl, heterocycloalkylalkyl-, aryl, arylalkyl-, heteroaryl, and heteroarylalkyl- is optionally substituted with 1-5 independently selected R²¹ substituents;

R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-5 independently selected R²¹ substituents;

R⁴, R⁶ and R⁷ are each independently selected from the group consisting of H, alkyl-, alkenyl-, alkynyl-, aryl-, arylalkyl-, alkylaryl-, cycloalkyl-, cycloalkenyl, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 independently selected R²¹ substituents;

R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-3 independently selected R²¹ substituents;

R⁹ is selected from the group consisting of alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-3 independently selected R²¹ substituents,

R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl-,

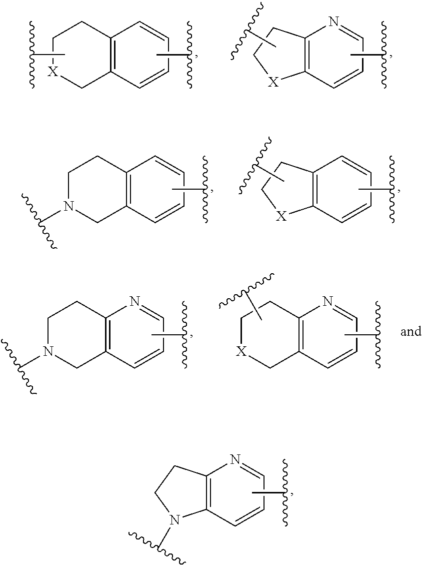

where X is O, N(R¹⁴) or S;

wherein each of said R¹⁰ groups is optionally substituted with 1-3 independently R²¹ substituents;

R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶); wherein each of said alkyl, alkenyl, alkynyl, cycloalkyl, cycloalkenyl, cycloalkylalkyl, cycloalkenyl, heterocyclyl, heterocyclylalkyl, aryl, arylalkyl, heteroaryl, and heteroarylalkyl is optionally substituted with 1-5 independently selected R²¹ substituents;

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ᵣ-alkyl, (R¹⁸)ᵣ-cycloalkyl, (R¹⁸)ᵣ-cycloalkylalkyl, (R¹⁸)ᵣ-heterocyclyl, (R¹⁸)ᵣ-heterocyclylalkyl, (R¹⁸)ᵣ-aryl, (R¹⁸)ᵣ-arylalkyl, (R¹⁸)ᵣ-heteroaryl and (R¹⁸)ᵣ-heteroarylalkyl, wherein each r is independently 1 to 5;

Each R¹⁸ is independently selected from the group consisting of: alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

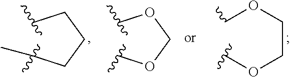

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

Each R²¹ is independently selected from the group consisting of: alkyl, alkenyl, alkynyl, cycloalkyl, cycloalkylalkyl-, cycloalkenyl, heterocycloalkyl, heterocycloalkylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ is optionally substituted by 1 to 5 independently selected R²² groups; and

Each R²² is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In one embodiment, the present application discloses a compound, or pharmaceutically acceptable salts, solvates, esters or prodrugs of said compound, said compound having the general structure shown in Formula I:

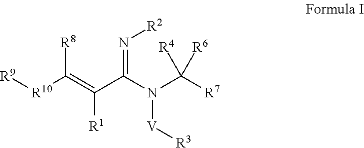

wherein:

either (i) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (ii) R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; or (iii) R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; and R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;

and

V is selected from the group consisting of a bond, O and N(R¹⁴);

R¹ (when R¹ is not joined to R³), is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is independently selected from the group consisting of H, alkyl, cycloalkyl, cycloalkylalkyl, heterocycloalkyl, heterocycloalkylalkyl, aryl, arylalkyl, heteroaryl, heteroarylalkyl, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵;

R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁴, R⁶ and R⁷ can be the same or different, each being independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

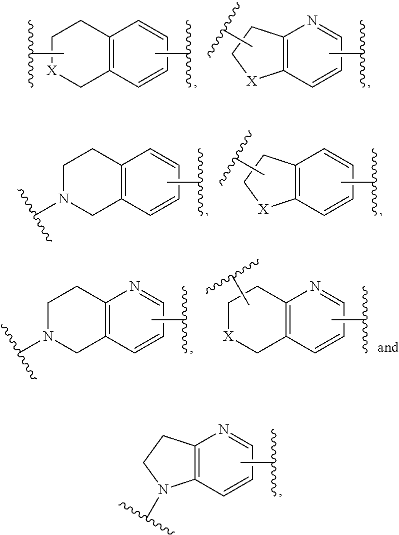

where X is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

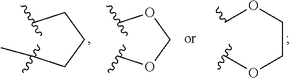

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocycloalkyl, heterocycloalkylalkyl, aryl, arylalkyl, heteroaryl, heteroarylalkyl, alkenyl and alkynyl groups in R¹, R², R³, R⁴, R⁶, R⁷, R⁸, R⁹, R¹⁰ and R¹⁴, or the 5-8 membered heterocyclyl or 5-8 membered heterocyclenyl moieties formed from the joining of R¹ and R³ or R³ and R⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

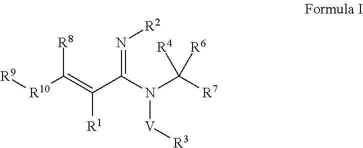

wherein:
R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;

V is selected from the group consisting of a bond, —O—, —S(O₂)—, —S(O)—, —C(O)—, and —N(R¹⁴)—;

R¹ (when R¹ is not joined to R³), is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is independently selected from the group consisting of H, alkyl, cycloalkyl, cycloalkenyl, cycloalkylalkyl, heterocycloalkyl, heterocycloalkylalkyl, aryl, arylalkyl, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalkyl, heterocycloalkyl, heterocycloalkylalkyl-, aryl, arylalkyl-, heteroaryl, and heteroarylalkyl- can be unsubstituted or optionally independently substituted with 1-5 substituents which can be the same or different, each substituent being independently selected from the group consisting of the moieties shown below;

R³ (when R³ is not joined to R¹), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁴, R⁶ and R⁷ can be the same or different, each being independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R¹⁴ is selected from the group consisting of H, alkyl, alkenyl, alkynyl, cycloalkyl, cycloalkenyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), C(═NOR¹⁵)R¹⁶, and —P(O)(OR¹⁵)(OR¹⁶), wherein each of said alkyl, alkenyl, alkynyl, cycloalkyl, cycloalkenyl, cycloalkylalkyl, heterocyclyl, heterocyclylalkyl, aryl, arylalkyl, heteroaryl, and heteroarylalkyl can be unsubstituted or optionally independently substituted with 1-5 substituents which can be the same or different, each substituent being independently selected from the group consisting of consisting of the moieties shown below;

R⁹ is selected from the group consisting of alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

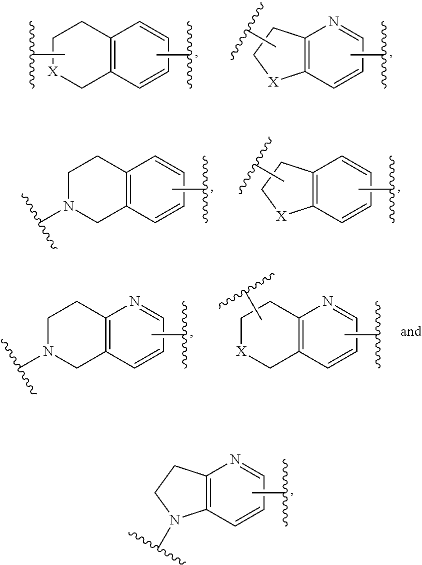

where X is O, N(R¹⁴) or S;

wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

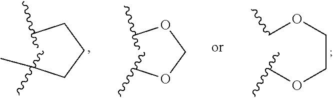

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocycloalkyl, heterocycloalkylalkyl, aryl, arylalkyl, heteroaryl, heteroarylalkyl, alkenyl and alkynyl groups in R¹, R², R³, R⁴, R⁶, R⁷, R⁸, R⁹, R¹⁰ and R¹⁴, or the 5-8 membered heterocyclyl or 5-8 membered heterocyclenyl moieties formed from the joining of R¹ and R³ or R³ and R⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

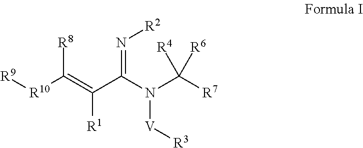

wherein:
R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;

V is selected from the group consisting of a bond, —O—, —S(O₂)—, —S(O)—, —C(O)—, and —N(R¹⁴)—;

R¹ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is independently selected from the group consisting of H, alkyl, cycloalkyl, cycloalkenyl, cycloalkylalkyl, heterocycloalkyl, heterocycloalkylalkyl, aryl, arylalkyl, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶)—S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalkyl, heterocycloalkyl, heterocycloalkylalkyl-, aryl, arylalkyl-, heteroaryl, and heteroarylalkyl- can be unsubstituted or optionally independently substituted with 1-5 substituents which can be the same or different, each substituent being independently selected from the group consisting of the moieties shown below;

R³ (when R³ is not joined to R⁴), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁴ (when R⁴ is not joined to R³), R⁶ and R⁷ can be the same or different, each being independently selected from the group consisting of H, alkyl-, alkenyl, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R¹⁴ is selected from the group consisting of H, alkyl, alkenyl, alkynyl, cycloalkyl, cycloalkenyl, cycloalkylalkyl-, cycloalkenyl, heterocyclyl, heterocyclylalkyl-, aryl, arylalkyl, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkenyl, cycloalkylalkyl-, heterocyclyl, heterocyclylalkyl-, aryl, arylalkyl-, heteroaryl, and heteroarylalkyl- can be unsubstituted or optionally independently substituted with 1-5 substituents which can be the same or different, each substituent being independently selected from the group consisting of consisting of the moieties shown below;

R⁹ is selected from the group consisting of alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

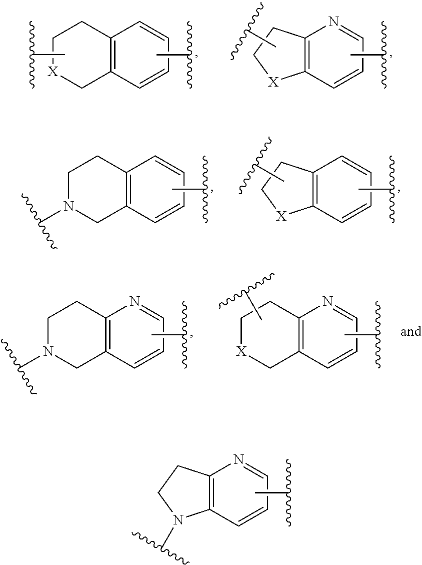

where X is O, N(R¹⁴) or S;

wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁶-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

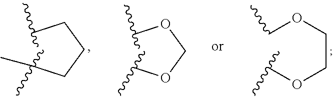

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkenyl, cycloalkylalkyl, heterocycloalkyl, heterocycloalkylalkyl, aryl, arylalkyl, heteroaryl, heteroarylalkyl, alkenyl and alkynyl groups in R¹, R², R³, R⁴, R⁶, R⁷, R⁸, R⁹, R¹⁹ and R¹⁴, or the 5-8 membered heterocyclyl or 5-8 membered heterocyclenyl moieties formed from the joining of R¹ and R³ or R³ and R⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

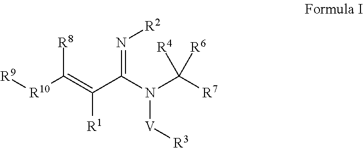

wherein:

R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; and R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;

and

V is selected from the group consisting of a bond, —O—, —S(O₂)—, —S(O)—, —C(O)—, and —N(R¹⁴)—;

R¹ (when R¹ is not joined to R³), is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is independently selected from the group consisting of H, alkyl, cycloalkyl, cycloalkenyl, cycloalkylalkyl-, heterocycloalkyl, heterocycloalkylalkyl-, aryl, arylalkyl-, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalkyl, heterocycloalkyl, heterocycloalkylalkyl-, aryl, arylalkyl-, heteroaryl, and heteroarylalkyl- can be unsubstituted or optionally independently substituted with 1-5 substituents which can be the same or different, each substituent being independently selected from the group consisting of the moieties shown below;

R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁴ (when R⁴ is not joined to R³), R⁶ and R⁷ can be the same or different, each being independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R¹⁴ is selected from the group consisting of H, alkyl, alkenyl, alkynyl, cycloalkyl, cycloalkenyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁶) R¹⁶, and —P(O)(OR¹⁵)(OR¹⁶), wherein each of said alkyl, alkenyl, alkynyl, cycloalkyl, cycloalkenyl, cycloalkylalkyl-, heterocyclyl, heterocyclylalkyl-, aryl, arylalkyl-, heteroaryl, and heteroarylalkyl- can be unsubstituted or optionally independently substituted with 1-5 substituents which can be the same or different, each substituent being independently selected from the group consisting of consisting of the moieties shown below;

R⁹ is selected from the group consisting of alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

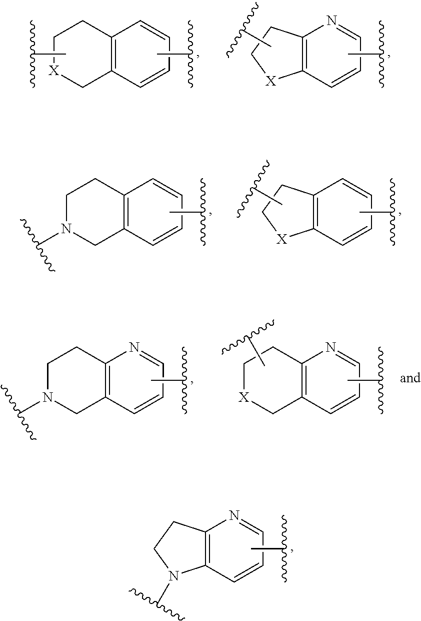

where X is O, N(R¹⁴) or S;

wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

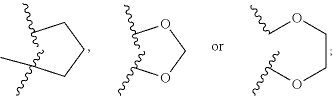

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocycloalkyl, heterocycloalkylalkyl, aryl, arylalkyl, heteroaryl, heteroarylalkyl, alkenyl and alkynyl groups in R¹, R², R³, R⁴, R⁶, R⁷, R⁸, R⁹, R¹⁰ and R¹⁴, or the 5-8 membered heterocyclyl or 5-8 membered heterocyclenyl moieties formed from the joining of R¹ and R³ or R³ and R⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment V is selected from the group consisting of a bond, —O—, and —N(R¹⁴)—;

In another embodiment R¹ (when R¹ is not joined to R³), is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown above (that is the moieties defined for R²¹); and R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown above (that is the moieties defined for R²¹), and the remaining substitutents are as defined for formula I.

In another embodiment R² is independently selected from the group consisting of H, alkyl, cycloalkyl, cycloalkylalkyl, heterocycloalkyl, heterocycloalkylalkyl-, aryl, arylalkyl-, heteroaryl, heteroarylalkyl-, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵.

In another embodiment R² is independently selected from the group consisting of H, alkyl, cycloalkyl, cycloalkylalkyl, heterocycloalkyl, heterocycloalkylalkyl-, aryl, arylalkyl-, heteroaryl, heteroarylalkyl-, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkylalkyl, heterocycloalkyl, heterocycloalkylalkyl-, aryl, arylalkyl-, heteroaryl, and heteroarylalkyl- can be unsubstituted or optionally independently substituted with 1-5 substituents which can be the same or different, each substituent being independently selected from the group consisting of the moieties shown above (that is the moieties defined for R²¹), and the remaining substitutents are as defined for formula I.

In another embodiment R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 independently selected R²¹ substituents.

In another embodiment R⁴, R⁶ and R⁷ are each independently selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 independently selected R²¹ substituents.

In another embodiment R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 independently selected R²¹ substituents.

In another embodiment R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 independently selected R²¹ substituents.

In another embodiment R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶).

In another embodiment R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶); wherein each of said alkyl, alkenyl, alkynyl, cycloalkyl, cycloalkylalkyl, cycloalkenyl, heterocyclyl, heterocyclylalkyl, aryl, arylalkyl, heteroaryl, and heteroarylalkyl is optionally substituted with 1-5 independently selected R²¹ substituents.

In another embodiment:
(a) R¹ (when R¹ is not joined to R³), is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown above (that is the moieties defined for R²¹);
(b) R² is independently selected from the group consisting of H, alkyl, cycloalkyl, cycloalkylalkyl, heterocycloalkyl, heterocycloalkylalkyl-, aryl, arylalkyl-, heteroaryl, heteroarylalkyl-, —CN, —OR¹⁵, —C(O)R¹⁵, —C(O)OR¹⁵, —C(O)N(R¹⁵)(R¹⁶), S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵.
(c) R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 independently selected R²¹ substituents;
(d) R⁴, R⁶ and R⁷ are each independently selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 independently selected R²¹ substituents;

(e) R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 independently selected R²¹ substituents;
(f) R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 independently selected R²¹ substituents;
(g) R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶); and
(h) In another embodiment V is selected from the group consisting of a bond, —O—, and —N(R¹⁴)—.

In another embodiment:
(a) R¹ (when R¹ is not joined to R³), is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown above (that is the moieties defined for R²¹);
(b) R² is independently selected from the group consisting of H, alkyl, cycloalkyl, cycloalkylalkyl, heterocycloalkyl, heterocycloalkylalkyl-, aryl, arylalkyl-, heteroaryl, heteroarylalkyl-, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkylalkyl, heterocycloalkyl, heterocycloalkylalkyl-, aryl, arylalkyl-, heteroaryl, and heteroarylalkyl- is optionally substituted with 1-5 independently selected R²¹ substituents;
(c) R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 independently selected R²¹ substituents;
(d) R⁴, R⁶ and R⁷ are each independently selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 independently selected R²¹ substituents;
(e) R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 independently selected R²¹ substituents;
(f) R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 independently selected R²¹ substituents;
(g) R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶); wherein each of said alkyl, alkenyl, alkynyl, cycloalkyl, cycloalkylalkyl, cycloalkenyl, heterocyclyl, heterocyclylalkyl, aryl, arylalkyl, heteroaryl, and heteroarylalkyl is optionally substituted with 1-5 independently selected R²¹ substituents; and
(h) In another embodiment V is selected from the group consisting of a bond, —O—, and —N(R¹⁴)—.

In another embodiment the optional ring described (i), (ii) or (iii) of formula is not present.

In another embodiment the optional ring described (i), (ii) or (iii) of formula is not present, V is a bond, and R³ is H.

In another embodiment the optional ring described (i), (ii) or (iii) of formula is not present, V is a bond, and R³ is alkyl (e.g., methyl).

In another embodiment the optional ring described (i), (ii) or (iii) of formula is not present, V is a bond, and R³ is alkyl substituted with 1 to 5 R²¹ groups.

In another embodiment the optional ring described (i), (ii) or (iii) of formula is not present, V is a bond, and R³ is alkyl substituted with 1 R²¹ group.

In another embodiment the optional ring described (i), (ii) or (iii) of formula is not present, V is a bond, and R³ is alkyl substituted with 1 R²¹ group, and said R²¹ group is —OR¹⁵.

In another embodiment the optional ring described (i), (ii) or (iii) of formula I is not present, V is a bond, and R³ is alkyl (e.g., propyl) substituted with 1 R²¹ group, and said R²¹ group is —OR¹⁵, and said R¹⁵ is alkyl (e.g., R¹⁵ is methyl).

In another embodiment the optional ring described (i), (ii) or (iii) of formula is not present, and R² is —OR¹⁵.

In another embodiment the optional ring described (i), (ii) or (iii) of formula is not present, R² is —OR¹⁵, and R¹⁵ is H.

In another embodiment the optional ring described (i), (ii) or (iii) of formula is not present, R² is —OR¹⁵, and R¹⁵ is alkyl (e.g., methyl).

In another embodiment the optional ring described (i), (ii) or (iii) of formula is not present, V is a bond, R³ is H, and R² is —OR¹⁵.

In another embodiment the optional ring described (i), (ii) or (iii) of formula is not present, V is a bond, R³ is alkyl (e.g., methyl), and R² is —OR¹⁵.

In another embodiment the optional ring described (i), (ii) or (iii) of formula is not present, V is a bond, R³ is alkyl substituted with 1 to 5 R²¹ groups, and R² is —OR¹⁵

In another embodiment the optional ring described (i), (ii) or (iii) of formula I is not present, V is a bond, R³ is alkyl substituted with 1 R²¹ group, and R² is —OR¹⁵

In another embodiment the optional ring described (i), (ii) or (iii) of formula I is not present, V is a bond, R³ is alkyl substituted with 1 R²¹ group, and said R²¹ group is —OR¹⁵, and R² is OR¹⁵.

In another embodiment the optional ring described (i), (ii) or (iii) of formula I is not present, V is a bond, and R³ is alkyl (e.g., propyl) substituted with 1 R²¹ group, and said R²¹ group is —OR¹⁵, said R¹⁵ is alkyl (e.g., R¹⁵ is methyl), and R² is —OR¹⁵

In another embodiment the optional ring described (i), (ii) or (iii) of formula I is not present, V is a bond, R³ is H, R² is —OR¹⁵, and R¹⁵ is H.

In another embodiment the optional ring described (i), (ii) or (iii) of formula I is not present, V is a bond, R³ is alkyl (e.g., methyl), R² is —OR¹⁵, and R¹⁵ is H.

In another embodiment the optional ring described (i), (ii) or (iii) of formula I is not present, V is a bond, R³ is alkyl substituted with 1 to 5 R²¹ groups, R² is OR¹⁵, and R¹⁵ is H.

In another embodiment the optional ring described (i), (ii) or (iii) of formula I is not present, V is a bond, R³ is alkyl substituted with 1 R²¹ group, R² is —OR¹⁵, and R¹⁵ is H.

In another embodiment the optional ring described (i), (ii) or (iii) of formula I is not present, V is a bond, R³ is alkyl substituted with 1 R²¹ group, and said R²¹ group is —OR¹⁵, R² is —OR¹⁵, and R¹⁵ is H.

In another embodiment the optional ring described (i), (ii) or (iii) of formula I is not present, V is a bond, and R³ is alkyl (e.g., propyl) substituted with 1 R²¹ group, and said R²¹ group is —OR¹⁵, said R¹⁵ is alkyl (e.g., R¹⁵ is methyl), R² is —OR¹⁵, and R¹⁵ is H.

In another embodiment the optional ring described (i), (ii) or (iii) of formula I is not present, V is a bond, R³ is H, R² is —OR¹⁵, and R¹⁵ is alkyl (e.g., methyl).

In another embodiment the optional ring described (i), (ii) or (iii) of formula I is not present, V is a bond, R³ is alkyl (e.g., methyl), R² is —OR¹⁵, and R¹⁵ is alkyl (e.g., methyl).

In another embodiment the optional ring described (i), (ii) or (iii) of formula I is not present, V is a bond, R³ is alkyl substituted with 1 to 5 R²¹ groups, R² is —OR¹⁵, and R¹⁵ is alkyl (e.g., methyl).

In another embodiment the optional ring described (i), (ii) or (iii) of formula I is not present, V is a bond, R³ is alkyl substituted with 1 R²¹ group, R² is —OR¹⁵, and R¹⁵ is alkyl (e.g., methyl).

In another embodiment the optional ring described (i), (ii) or (iii) of formula I is not present, V is a bond, R³ is alkyl substituted with 1 R²¹ group, and said R²¹ group is —OR¹⁵, R² is —OR¹⁵, and R¹⁵ is alkyl (e.g., methyl).

In another embodiment the optional ring described (i), (ii) or (iii) of formula I is not present, V is a bond, and R³ is alkyl (e.g., propyl) substituted with 1 R²¹ group, and said R²¹ group is —OR¹⁵, said R¹⁵ is alkyl (e.g., R¹⁵ is methyl), R² is —OR¹⁵, and R¹⁵ is alkyl (e.g., methyl).

In another embodiment the optional ring described (i), (ii) or (iii) of formula I is not present, and: (a) V is a bond; (b) R² is selected from the group consisting of: —OR¹⁵, —OR¹⁵ wherein R¹⁵ is H, —OR¹⁵ wherein R¹⁵ is alkyl; and (c) R³ is selected from the group consisting of: H, is alkyl, alkyl substituted with 1 to 5 R²¹ groups, alkyl substituted with 1 R²¹ group, alkyl substituted with —OR¹⁵, and alkyl substituted with —OR¹⁵ wherein R¹⁵ is alkyl.

In another embodiment the optional ring described in (i), (ii), or (iii) of formula I is present.

In another embodiment the optional ring described in (i) of formula I is present.

In another embodiment the optional ring described in (ii) of formula I is present.

In another embodiment the optional ring described in (iii) of formula I is present.

In another embodiment R¹ and R³ are joined together to form a 5-8 membered heterocyclyl moiety that is optionally substituted with 1-5 independently selected R²¹ groups.

In another embodiment R¹ and R³ are joined together to form a 5-8 membered heterocyclyl moiety that is optionally fused with an aryl ring or a heteroaryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally fused with an aryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally fused with a heteroaryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups.

In another embodiment R¹ and R³ are joined together to form a 5-8 membered heterocyclenyl moiety that is optionally fused with an aryl ring or a heteroaryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally fused with an aryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally fused with a heteroaryl ring wherein the ring moiety resulting from the fusion is optionally substituted with 1-5 independently selected R²¹ groups.

In another embodiment R³ and R⁴ are joined together to form a 5-8 membered heterocyclyl moiety that is optionally substituted with 1-5 independently selected R²¹ groups.

In another embodiment R³ and R⁴ are joined together to form a 5-8 membered heterocyclyl moiety that is optionally fused with an aryl ring or a heteroaryl ring wherein the ring moiety resulting from the fusion is optionally substituted with 1-5 independently selected R²¹ groups.

In another embodiment R³ and R⁴ are joined together to form a 5-8 membered heterocyclyl moiety that is optionally fused with an aryl ring wherein the ring moiety resulting from the fusion is optionally substituted with 1-5 independently selected R²¹ groups.

In another embodiment R³ and R⁴ are joined together to form a 5-8 membered heterocyclyl moiety that is optionally fused with a heteroaryl ring wherein the ring moiety resulting from the fusion is optionally substituted with 1-5 independently selected R²¹ groups.

In another embodiment R³ and R⁴ are joined together to form a 5-8 membered heterocyclenyl moiety that is optionally substituted with 1-5 independently selected R²¹ groups.

In another embodiment R³ and R⁴ are joined together to form a 5-8 membered heterocyclenyl moiety that is optionally fused with an aryl ring or a heteroaryl ring wherein the ring moiety resulting from the fusion is optionally substituted with 1-5 independently selected R²¹ groups.

In another embodiment R³ and R⁴ are joined together to form a 5-8 membered heterocyclenyl moiety that is optionally fused with an aryl ring wherein the ring moiety resulting from the fusion is optionally substituted with 1-5 independently selected R²¹ groups.

In another embodiment R³ and R⁴ are joined together to form a 5-8 membered heterocyclenyl moiety that is optionally fused with a heteroaryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups.

In another embodiment R¹ and R³ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and R³ and R⁴ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups, and wherein the ring moiety resulting from R¹ and R³ being taken together and R³ and R⁴ being taken together is optionally fused with an aryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups, and wherein the ring moiety resulting from R¹ and R³ being taken together and R³ and R⁴ being taken together is optionally fused with a heteroaryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl ring wherein the ring moiety resulting from the fusion is optionally substituted with 1-5 independently selected R²¹ groups.

In another embodiment R¹ and R³ are joined together to form a 5-8 membered heterocyclyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with a heteroaryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups, and wherein the ring moiety resulting from R¹ and R³ being taken together and R³ and R⁴ being taken together is optionally fused with an aryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclyl moiety that is optionally substituted with 1-5 independently selected R²¹ groups, and wherein the ring moiety resulting from R¹ and R³ being taken together and R³ and R⁴ being taken together is optionally fused with a heteroaryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl ring wherein the ring moiety resulting from the fusion is optionally substituted with 1-5 independently selected R²¹ groups.

In another embodiment R¹ and R³ are joined together to form a 5-8 membered heterocyclenyl moiety that is optionally substituted with 1-5 independently selected R²¹ groups, and R³ and R⁴ are joined together to form a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with a heteroaryl ring wherein the ring moiety resulting from the fusion is optionally substituted with 1-5 independently selected R²¹ groups.

In another embodiment, R⁸ is H.

In another embodiment, R⁸ is alkyl.

In another embodiment, R⁸ is methyl.

In another embodiment, R¹ and R³ are joined together to form a heterocyclyl moiety.

In another embodiment, R¹ and R³ are joined together to form a heterocyclenyl moiety.

In another embodiment, R³ and R⁴ are joined together to form a heterocyclyl moiety.

In another embodiment, R³ and R⁴ are joined together to form a heterocyclenyl moiety.

In another embodiment, R¹ and R³ are joined together to form a heterocyclyl or heterocyclenyl moiety and R³ and R⁴ are joined together to form a heterocyclyl or heterocyclenyl moiety.

In another embodiment, R¹ and R³ are joined together to form a ring selected from the group consisting of:

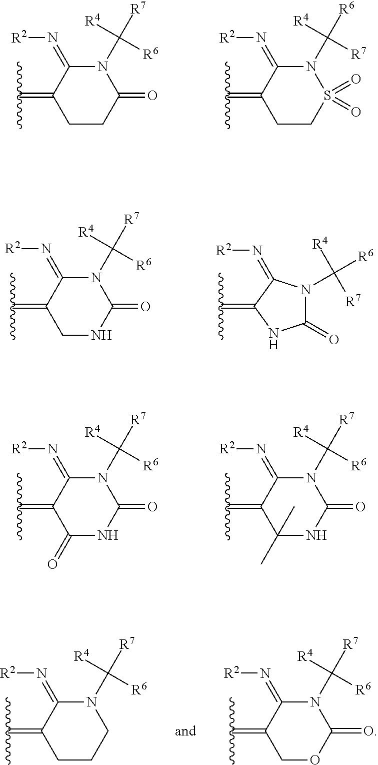

In another embodiment, R¹ and R³ are joined together to form a ring selected from the group consisting of:

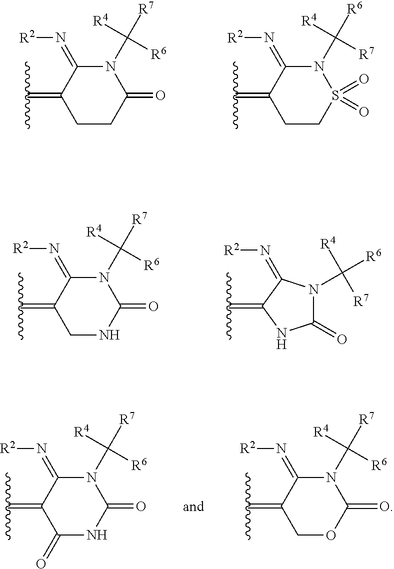

In another embodiment, R¹ and R³ are joined together to form

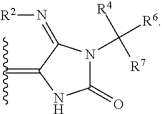

In another embodiment, R¹ and R³ are joined together to form

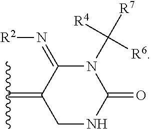

In another embodiment, R¹ and R³ are joined together to form

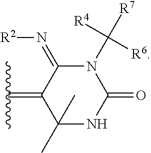

In another embodiment, R¹ and R³ are joined together to form

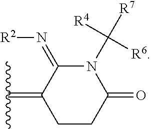

In another embodiment, R¹ and R³ are joined together to form

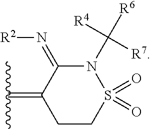

In another embodiment, R¹ and R³ are joined together to form

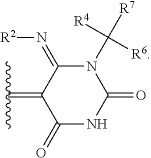

In another embodiment, R¹ and R³ are joined together to form

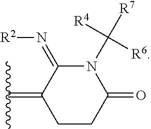

In another embodiment, R¹ and R³ are joined together to form

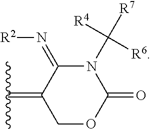

In another embodiment, R³ and R⁴ are joined together to form a moiety selected from the group consisting of:

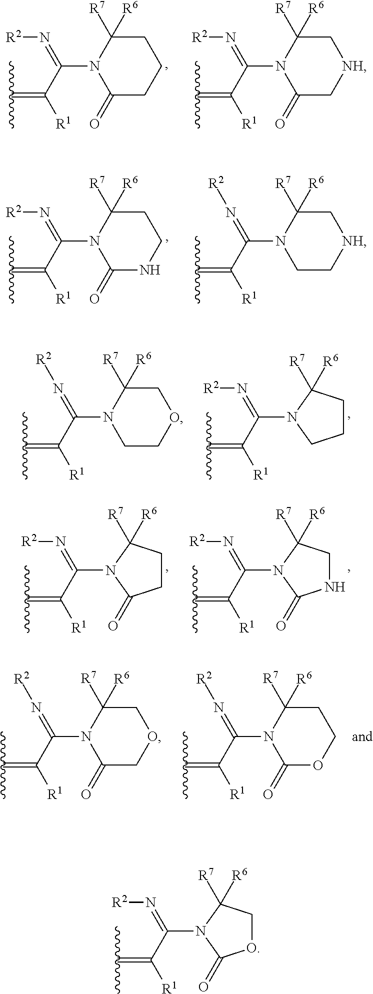

In another embodiment, R³ and R⁴ are joined together to form

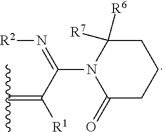

In another embodiment, R³ and R⁴ are joined together to form

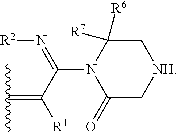

In another embodiment, R³ and R⁴ are joined together to form

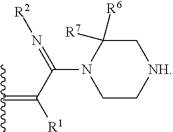

In another embodiment, R³ and R⁴ are joined together to form

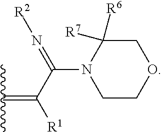

In another embodiment, R³ and R⁴ are joined together to form

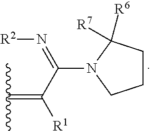

In another embodiment, R³ and R⁴ are joined together to form

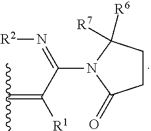

In another embodiment, R³ and R⁴ are joined together to form

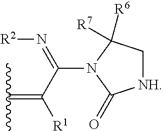

In another embodiment, R³ and R⁴ are joined together to form

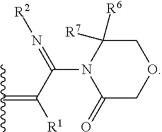

In another embodiment, R³ and R⁴ are joined together to form

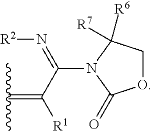

In another embodiment, R³ and R⁴ are joined together to form

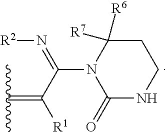

In another embodiment, R³ and R⁴ are joined together to form

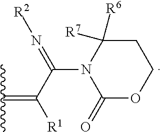

In another embodiment, R¹ and R³ are joined together and R³ and R⁴ are joined together to form a moiety selected from the group consisting of:

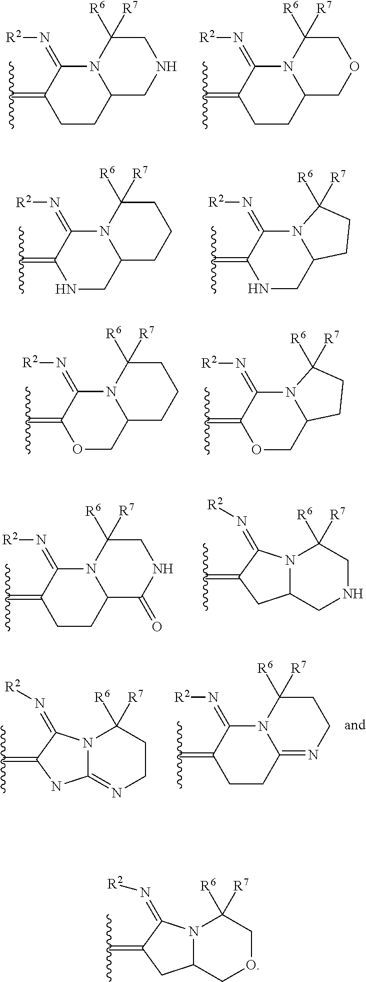

In another embodiment, R¹ and R³ are joined together and R³ and R⁴ are joined together to form

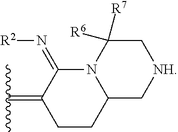

In another embodiment, R¹ and R³ are joined together and R³ and R⁴ are joined together to form

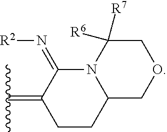

In another embodiment, R¹ and R³ are joined together and R³ and R⁴ are joined together to form

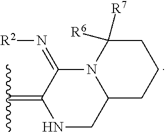

In another embodiment, R¹ and R³ are joined together and R³ and R⁴ are joined together to form

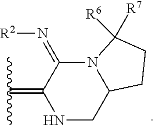

In another embodiment, R¹ and R³ are joined together and R³ and R⁴ are joined together to form

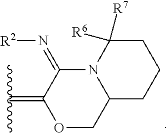

In another embodiment, R¹ and R³ are joined together and R³ and R⁴ are joined together to form

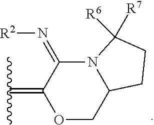

In another embodiment, R¹ and R³ are joined together and R³ and R⁴ are joined together to form

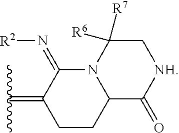

In another embodiment, R¹ and R³ are joined together and R³ and R⁴ are joined together to form

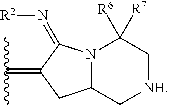

In another embodiment, R¹ and R³ are joined together and R³ and R⁴ are joined together to form

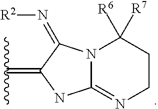

In another embodiment, R¹ and R³ are joined together and R³ and R⁴ are joined together to form

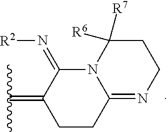

In another embodiment, R¹ and R³ are joined together and R³ and R⁴ are joined together to form

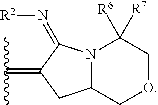

In another embodiment, R⁴, R⁶ and R⁷ can be the same or different, each being independently selected from the group consisting of H, alkyl and aryl-.

In another embodiment, R⁴, R⁶ and R⁷ can be the same or different, each being independently selected from the group consisting of H, methyl and

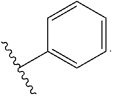

In another embodiment, R⁴, R⁶ and R⁷ can be the same or different, each being independently selected from the group consisting of H, alkyl and aryl-, wherein said aryl- is substituted with 1-3 halogen.

In another embodiment, R⁴, R⁶ and R⁷ can be the same or different, each being independently selected from the group consisting of H, methyl and

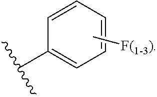

In another embodiment, R² is H, alkyl, —OH or alkoxy-.

In another embodiment, R² is H.

In another embodiment, R² is —OH.

In another embodiment, R² is alkoxy.

In another embodiment, R² is methyloxy-.

In another embodiment, R² is ethyloxy-.

In another embodiment, R² is alkyl-.

In another embodiment, R² is methyl.

In another embodiment, R¹⁰ is aryl- and said aryl- is unsubstituted.

In another embodiment, R¹⁰ is

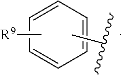

In another embodiment, R¹⁰ is aryl- and said aryl- is substituted with 1-3 substitutents, which can be the same or different, each being independently selected from the group consisting of halo, alkyl, —CN, —NH₂, —NH(alkyl), —N(alkyl)₂, hydroxy and alkoxy groups.

In another embodiment, R¹⁰ is

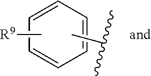

R¹⁰ is substituted with 1-3 substitutents, which can be the same or different, each being independently selected from the group consisting of halo, alkyl, CN, NH₂, NH(alkyl), N(alkyl)₂, hydroxy and alkoxy groups.

In another embodiment, R¹⁰ is unsubstituted heteroaryl.

In another embodiment, R¹⁰ is heteroaryl substituted with 1-3 substitutents, which can be the same or different, each being independently selected from the group consisting of halo, alkyl, CN, NH₂, NH(alkyl), N(alkyl)₂, hydroxy and alkoxy groups.

In another embodiment R¹⁰ is aryl substituted with 1 to 3 independently selected R²¹ moieties.

In another embodiment R¹⁰ is aryl substituted with 1 to 3 R²¹ moieties, wherein each R²¹ moiety is the same or different —OR¹⁵ group.

In another embodiment R¹⁰ is aryl substituted with 1 R²¹ moiety.

In another embodiment R¹⁰ is aryl substituted with one R²¹ moiety, and said R²¹ moiety is —OR¹⁵.

In another embodiment R¹⁰ is aryl substituted with one R²¹ moiety, said R²¹ moiety is —OR¹⁵, and said R¹⁵ is alkyl.

In another embodiment R¹⁰ is phenyl substituted with 1 to 3 independently selected R²¹ moieties.

In another embodiment R¹⁰ is phenyl substituted with 1 to 3 R²¹ moieties, wherein each R²¹ moiety is the same or different —OR¹⁵ group.

In another embodiment R¹⁰ is phenyl substituted with 1 R²¹ moiety.

In another embodiment R¹⁰ is phenyl substituted with one R²¹ moiety, and said R²¹ moiety is —OR¹⁵.

In another embodiment R¹⁰ is phenyl substituted with one R²¹ moiety, said R²¹ moiety is —OR¹⁵, and said R¹⁵ is alkyl.

In another embodiment R¹⁰ is:

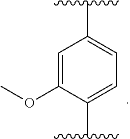

In another embodiment R¹⁰ is:

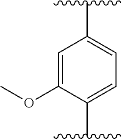

wherein the —R¹⁰-R⁹ moiety is:

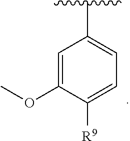

In another embodiment R¹⁰ is aryl substituted with 1 to 3 R²¹ moieties, wherein each R²¹ moiety is the same or different halo.

In another embodiment R¹⁰ is aryl substituted with 1 to 3 R²¹ moieties, wherein each R²¹ moiety is F.

In another embodiment R¹⁰ is aryl substituted with one R²¹ moiety, and said R²¹ moiety is halo.

In another embodiment R¹⁰ is aryl substituted with one R²¹ moiety, said R²¹ moiety is -halo, and said halo is F.

In another embodiment R¹⁰ is phenyl substituted with 1 to 3 R²¹ moieties, wherein each R²¹ moiety is the same or different halo.

In another embodiment R¹⁰ is phenyl substituted with 1 to 3 R²¹ moieties, wherein each R²¹ moiety is F.

In another embodiment R¹⁰ is phenyl substituted with one R²¹ moiety, and said R²¹ moiety is halo.

In another embodiment R¹⁰ is phenyl substituted with one R²¹ moiety, said R²¹ moiety is -halo, and said halo is F.

In another embodiment R¹⁰ is:

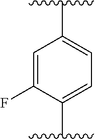

In another embodiment R¹⁰ is:

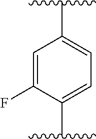

wherein the —R¹⁰-R⁹ moiety is:

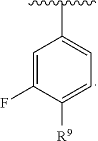

In another embodiment, R¹⁰ is unsubstituted heteroaryl.

In another embodiment R¹⁰ is unsubstituted heteroaryl wherein said heteroaryl is pyridyl.

In another embodiment R¹⁰ is:

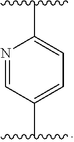

In another embodiment R¹⁰ is:

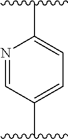

wherein the —R¹⁰-R⁹ moiety is:

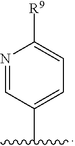

In another embodiment R¹⁰ is selected from the group consisting of:

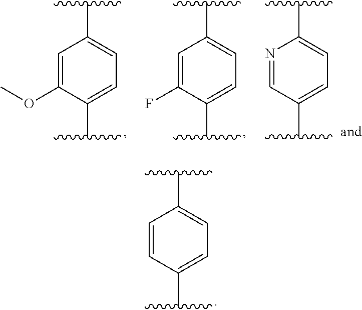

In another embodiment, R¹⁰ is aryl- and said aryl- is substituted with 1-3 substitutents, which can be the same or different, each being an alkoxy group.

In another embodiment, R¹⁰ is

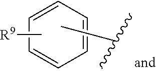

R¹⁰ is substituted with 1-3 substitutents, which can be the same or different, each being an alkoxy group.

In another embodiment, R¹⁰ is aryl-substituted with methoxy.

In another embodiment, R¹⁰ is

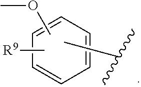

In another embodiment, R⁹ is unsubstituted heteroaryl.

In another embodiment of this invention R⁹ is selected from the group consisting of heteroaryl and heteroaryl substituted with 1-3 R²¹ groups, and wherein each R²¹ is independently selected.

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxyl, alkoxy, alkyl substituted with halo (e.g., alkyl substituted with F, such as, for example, —CH₂F), and alkyl substituted with —OR¹⁵ (such as, for example, alkyl substituted with —OR¹⁵ wherein R¹⁵ is H, that is, —CH₂OH).

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups.

In another embodiment of this invention R⁹ is selected from the group consisting of imidazolyl and imidazolyl substituted with 1-3 R²¹ groups, and wherein each R²¹ is independently selected.

In another embodiment of this invention R⁹ is imidazolyl substituted with 1-3 R²¹ groups, and wherein each R²¹ is independently selected.

In another embodiment, R⁹ is imidazolyl which is substituted with 1-3 substituents which can be the same or different, each substituent being independently selected from the group consisting of halo, alkyl, CN, NH₂, NH(alkyl), N(alkyl)₂, hydroxyl, alkoxy, alkyl substituted with halo (e.g., alkyl substituted with F, such as, for example, —CH₂F), and alkyl substituted with —OR¹⁵ (such as, for example, alkyl substituted with —OR¹⁵ wherein R¹⁵ is H, that is, —CH₂OH).

In another embodiment, R⁹ is imidazolyl substituted with 1-3 substituents independently selected from the group consisting of halo, alkyl, CN, NH₂,

NH(alkyl), N(alkyl)₂, hydroxy and alkoxy groups.

In another embodiment, R⁹ is imidazol-1-yl.

In another embodiment, R⁹ is 4-methyl-imidazol-1-yl.

In another embodiment, R⁹ is 5-chloro-4-methyl-imidazol-1-yl.

In another embodiment, R⁹ is:

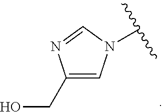

In another embodiment, R⁹ is:

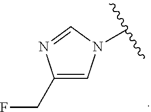

In another embodiment R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

In another embodiment R¹⁰ is selected from the group consisting of phenyl and phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is selected from the group consisting of heteroaryl and heteroaryl substituted with 1-3 R²¹ groups, and the R⁹ group is selected from the group consisting of heteroaryl and heteroaryl substituted with 1-3 R²¹ groups, and wherein each R²¹ is independently selected.

In another embodiment R¹⁰ is selected from the group consisting of pyridyl and pyridyl substituted with 1-3 R²¹ groups, and the R⁹ group is selected from the group consisting of imidazolyl and imidazolyl substituted with 1-3 R²¹ groups, and wherein each R²¹ is independently selected.

In another embodiment R¹⁰ is pyridyl, and the R⁹ group is imidazolyl substituted with 1-3 R²¹ groups, and wherein each R²¹ is independently selected.

In another embodiment the R⁹-R¹⁰— moiety is:

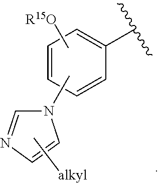

In another embodiment the R⁹-R¹⁰— moiety is:

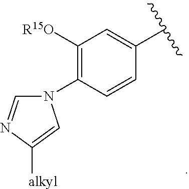

In another embodiment the R⁹— R¹⁰— moiety is:

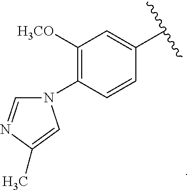

In another embodiment the R⁹-R¹⁰— moiety is:

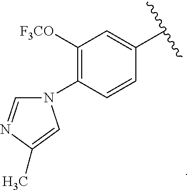

In another embodiment the R⁹-R¹⁰— moiety is:

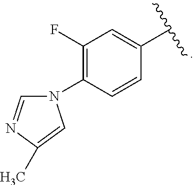

In another embodiment R⁹-R¹⁰— moiety is:

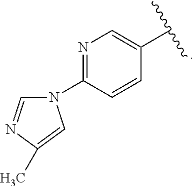

In another embodiment R⁹-R¹⁰— moiety is:

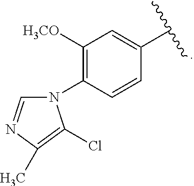

In another embodiment, the present application discloses a compound, or pharmaceutically acceptable salts, solvates, esters or prodrugs of said compound, said compound having the general structure shown in Formula:

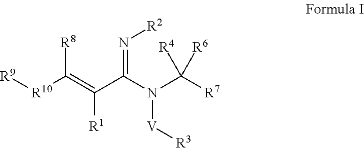

wherein:

R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;

V is selected from the group consisting of a bond, —O—, —S(O₂)—, —S(O)—, —C(O)—, and —N(R¹⁴)— (and in one example V is selected from the group consisting of a bond, O and N(R¹⁴));

R² is H, alkyl, —OH or alkoxy-;

R⁴, R⁶ and R⁷ can be the same or different, each being independently selected from the group consisting of H, alkyl and ary-I, wherein said aryl- is substituted with 1-3 halogen (e.g., 1-3 independently selected halogens);

R⁸ is H;

R⁹ is 4-methyl-imidazol-1-yl;

R¹⁰ is

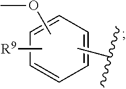

R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁶)(R¹⁶), —C(═NOR¹⁵)R¹⁶, and —P(O)(OR¹⁵)(OR¹⁶);

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

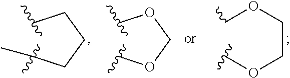

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein the 5-8 membered heterocyclyl or 5-8 membered heterocyclenyl moieties formed from the joining of R¹ and R³, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

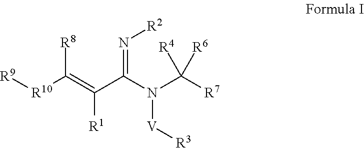

wherein:
R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;
   and

V is selected from the group consisting of a bond, O and N(R¹⁴);

R¹ is selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is H, alkyl, —OH or alkoxy-;

R⁶ and R⁷ can be the same or different, each being independently selected from the group consisting of H, alkyl and aryl-, wherein said aryl- is substituted with 1-3 halogen (e.g., 1-3 independently selected halogens);

R⁸ is H;

R⁹ is 4-methyl-imidazol-1-yl;
R¹⁰ is

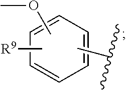

R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁸);

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

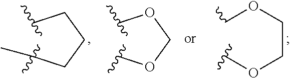

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkenyl, cycloalkylalkyl, heterocycloalkyl, heterocycloalkylalkyl, aryl, arylalkyl, heteroaryl, heteroarylalkyl, alkenyl and alkynyl groups in R¹, or the 5-8 membered heterocyclyl or 5-8 membered heterocyclenyl moieties formed from the joining of R³ and R⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

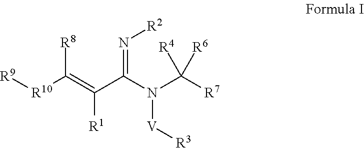

wherein:

R¹ and R³ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below; and R³ and R⁴ are joined together to form a 5-8 membered heterocyclyl or 5-8 membered heterocyclenyl moiety, with each of said heterocyclyl or heterocyclenyl moiety being unsubstituted or optionally independently being substituted with 1-5 substituents which can be the same or different, each substituent being independently selected from the group consisting of the moieties shown below;

and

V is selected from the group consisting of a bond, O and N(R¹⁴);

R² is H, alkyl, —OH or alkoxy-;

R⁶ and R⁷ can be the same or different, each being independently selected from the group consisting of H, alkyl and aryl-, wherein said aryl- is substituted with 1-3 halogen (e.g., 1-3 independently selected halogens);

R⁸ is H;

R⁹ is 4-methyl-imidazol-1-yl;

R¹⁰ is

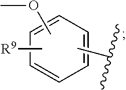

R¹⁴ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

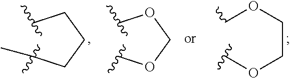

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein the 5-8 membered heterocyclyl or 5-8 membered heterocyclenyl moieties formed from the joining of R¹ and R³ and R³ and R⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

An illustrative group of compounds of the invention are shown in Table 1.

TABLE 1

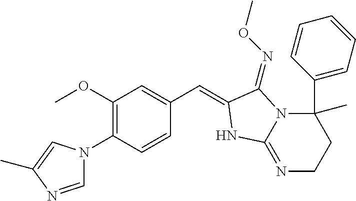

1

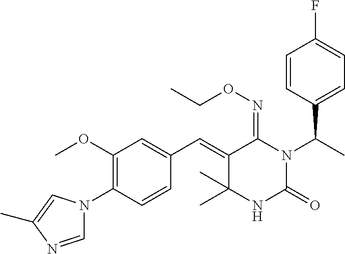

2

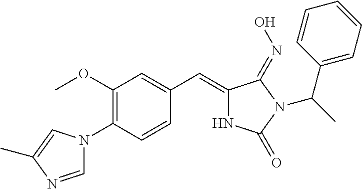

3

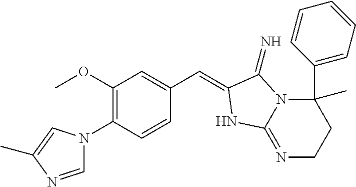

4

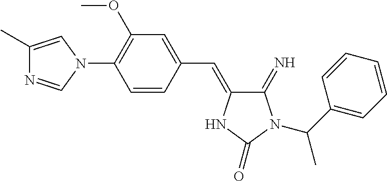

5

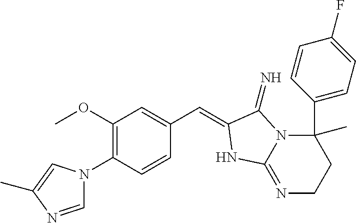

6

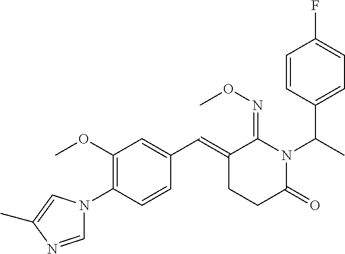

7

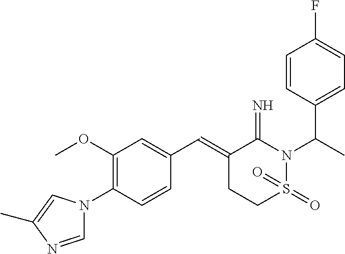

8

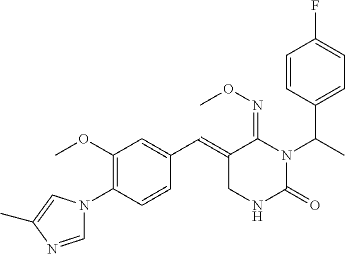

9

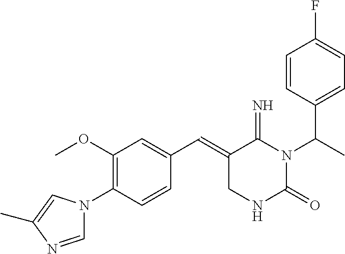

10

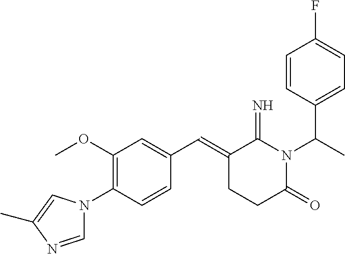

11

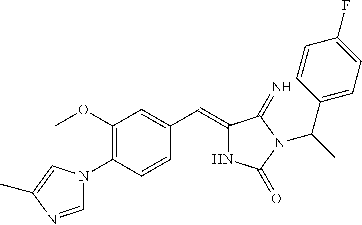

12

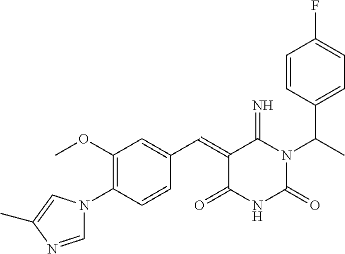

13

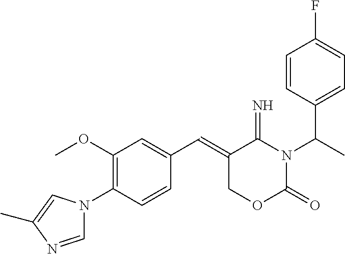

14

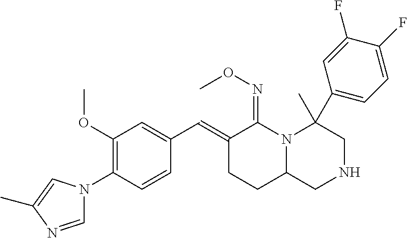

15

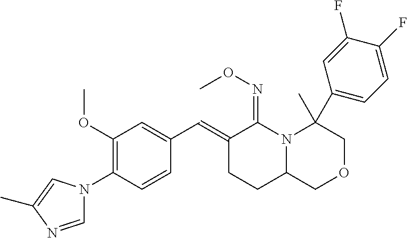

16

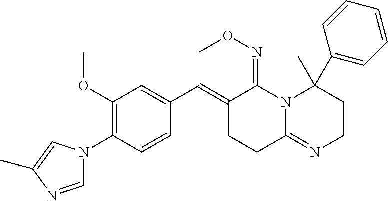

17

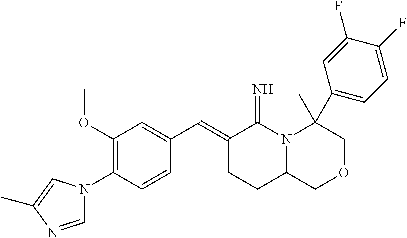

18

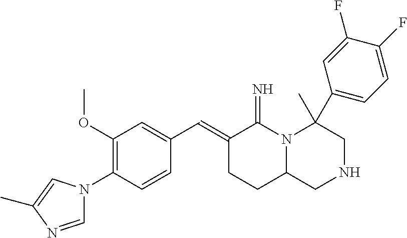

19

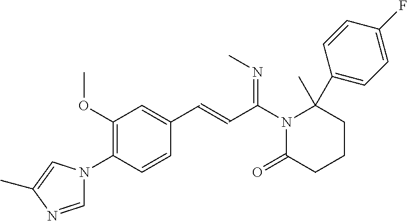

20

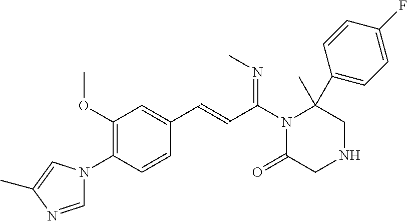

21

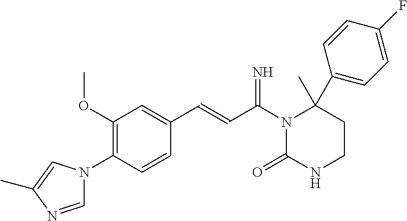

22

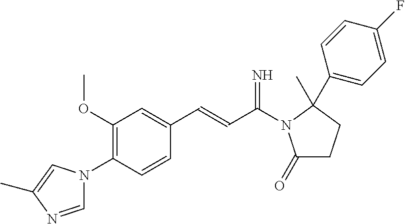

23

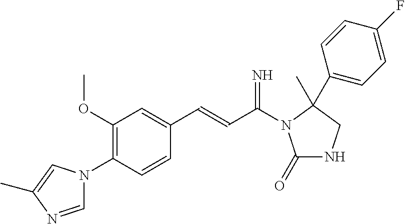

24

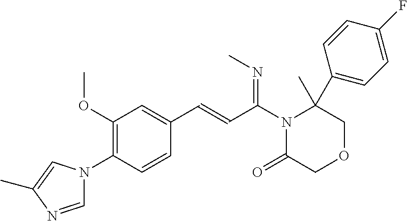

25

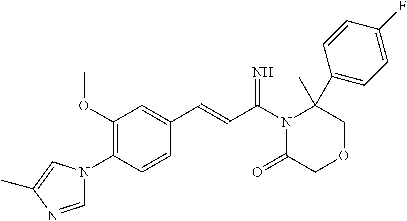

26

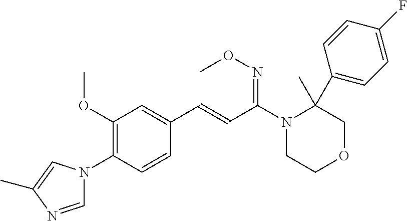

27

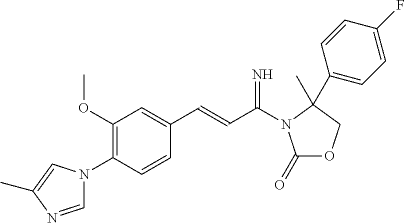

28

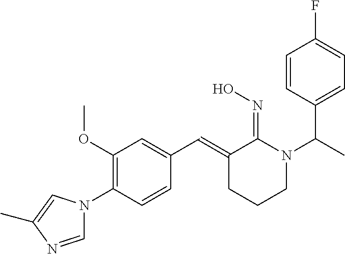

29

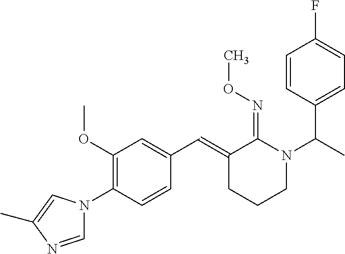

30

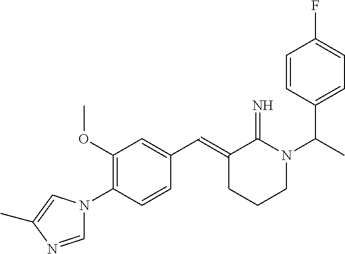

31

or a pharmaceutically acceptable salt, solvate, ester or prodrug thereof.

Representative compounds of the invention also include compounds 32 (see Method G), 33-40 (see Method H), 41-46 (see Method I), 47 (see Method J), and 48 (see Method K), or a pharmaceutically acceptable salt, solvate, ester or prodrug thereof.

In another embodiment the compound of formula I is selected from the group consisting of: compounds 1 to 48.

In another embodiment the compound of formula I is selected from the group consisting of: compounds 32 to 40.

In another embodiment the compound of formula I is compound 1.

In another embodiment the compound of formula I is compound 2.

In another embodiment the compound of formula I is compound 3.

In another embodiment the compound of formula I is compound 4.

In another embodiment the compound of formula I is compound 5.

In another embodiment the compound of formula I is compound 6.

In another embodiment the compound of formula I is compound 7.

In another embodiment the compound of formula I is compound 8.

In another embodiment the compound of formula I is compound 9.

In another embodiment the compound of formula I is compound 10.

In another embodiment the compound of formula I is compound 11.

In another embodiment the compound of formula I is compound 12.

In another embodiment the compound of formula I is compound 13.

In another embodiment the compound of formula I is compound 14.

In another embodiment the compound of formula I is compound 15.

In another embodiment the compound of formula I is compound 16.

In another embodiment the compound of formula I is compound 17.

In another embodiment the compound of formula I is compound 18.

In another embodiment the compound of formula I is compound 19.

In another embodiment the compound of formula I is compound 20.

In another embodiment the compound of formula I is compound 21.

In another embodiment the compound of formula I is compound 22.

In another embodiment the compound of formula I is compound 23.

In another embodiment the compound of formula I is compound 24.

In another embodiment the compound of formula I is compound 25.

In another embodiment the compound of formula I is compound 26.

In another embodiment the compound of formula I is compound 27.

In another embodiment the compound of formula I is compound 28.

In another embodiment the compound of formula I is compound 29.

In another embodiment the compound of formula I is compound 30.

In another embodiment the compound of formula I is compound 31.

In another embodiment the compound of formula I is compound 32.

In another embodiment the compound of formula I is compound 33.

In another embodiment the compound of formula I is compound 34.

In another embodiment the compound of formula I is compound 35.

In another embodiment the compound of formula I is compound 36.

In another embodiment the compound of formula I is compound 37.

In another embodiment the compound of formula I is compound 38.

In another embodiment the compound of formula I is compound 39.

In another embodiment the compound of formula I is compound 40.

In another embodiment the compound of formula I is compound 41.

In another embodiment the compound of formula I is compound 42.

In another embodiment the compound of formula I is compound 43.

In another embodiment the compound of formula I is compound 44.

In another embodiment the compound of formula I is compound 45.

In another embodiment the compound of formula I is compound 46.

In another embodiment the compound of formula I is compound 47.

In another embodiment the compound of formula I is compound 48.

As used above, and throughout this disclosure, the following terms, unless otherwise indicated, shall be understood to have the following meanings:

“Patient” includes both human and animals.

“Mammal” means humans and other mammalian animals.

It is noted that the carbons of formula I and other formulas herein may be replaced with 1 to 3 silicon atoms so long as all valency requirements are satisfied.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

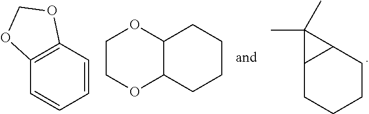

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” (or “heterocycloalkyl”) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a heterocyclyl ring wherein a single moiety (e.g., carbonyl) simultaneously replaces two available hydrogens on the same carbon atom on a ring system. An example of such moiety is pyrrolidone:

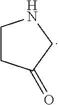

“Heterocyclylalkyl” (or “heterocycloalkyalkyl-”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

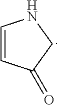

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

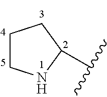

there is no —OH attached directly to carbons marked 2 and 5.

It should also be noted that tautomeric forms such as, for example, the moieties:

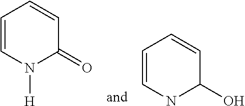

are considered equivalent in certain embodiments of this invention.

It should also be noted that tautomeric forms such as, for example, the moieties:

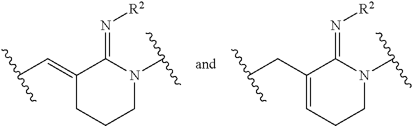

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C1-C2)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₈)alkanoyloxy)ethyl, 1-methyl-1-((C₁-C₈)alkanoyloxy)ethyl, (C₁-C₈)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula I, and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula I, and of the salts, solvates, esters and prodrugs of the compounds of Formula I, are intended to be included in the present invention.

The compounds according to the invention can have pharmacological properties; in particular, the compounds of Formula I can be modulators of gamma secretase (including inhibitors, antagonists and the like).

More specifically, the compounds of Formula I can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula I, or a pharmaceutically acceptable salt, solvate, ester or prodrug of said compound to the mammal.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula I, or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

Other embodiments of this invention are directed to pharmaceutically acceptable salts of any one of the embodiments above directed to any one of compounds 1 to 48.

Other embodiments of this invention are directed to pharmaceutically acceptable esters of any one of the embodiments above directed to any one of compounds 1 to 48.

Other embodiments of this invention are directed to solvates of any one of the embodiments above directed to any one of compounds 1 to 48.

One embodiment of this invention is directed to a compound of formula I.

Another embodiment of this invention is directed to a pharmaceutically acceptable salt of a compound of formula I.

Another embodiment of this invention is directed to a pharmaceutically acceptable ester of a compound of formula I.

Another embodiment of this invention is directed to a solvate of a compound of formula I.

Another embodiment of this invention is directed to a compound of formula I in isolated form.

Another embodiment of this invention is directed to a compound of formula I in pure form.

Another embodiment of this invention is directed to a compound of formula I in pure and isolated form.

Another embodiment of this invention is directed to a pharmaceutical composition comprising an effective amount of one or more (e.g., one) compounds of formula I and a pharmaceutically acceptable carrier.

Another embodiment of this invention is directed to a pharmaceutical composition comprising an effective amount of a pharmaceutically acceptable salt of one or more (e.g., one) compounds of formula I and a pharmaceutically acceptable carrier.

Another embodiment of this invention is directed to a pharmaceutical composition comprising an effective amount of a pharmaceutically acceptable ester of one or more (e.g., one) compounds of formula I and a pharmaceutically acceptable carrier.

Another embodiment of this invention is directed to a pharmaceutical composition comprising an effective amount of a solvate of one or more (e.g., one) compounds of formula I and a pharmaceutically acceptable carrier.

Another embodiment of this invention is directed to a pharmaceutical composition comprising an effective amount of one or more (e.g., one) compounds of formula I, and an effective amount of one or more (e.g., one) other pharmaceutically active ingredients (e.g.,) drugs, and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds of formula I, and effective amount of one or more BACE inhibitors, and a pharmaceutically acceptable carrier.

Another embodiment of this invention is directed to a pharmaceutical composition comprising an effective amount of one or more (e.g., one) compounds of formula I, and effective amount of one or more cholinesterase inhibitors (e.g., acetyl- and/or butyrylchlolinesterase inhibitors), and a pharmaceutically acceptable carrier.

The compounds of formula I can be useful as gamma secretase modulators and can be useful in the treatment and prevention of diseases such as, for example, central nervous system disorders such as Alzheimers disease and Downs Syndrome.

Thus, another embodiment of this invention is directed to a method for modulating (including inhibiting, antagonizing and the like) gamma-secretase comprising administering an effective amount of one or more (e.g., one) compounds of formula I to a patient in need of such treatment.

Another embodiment of this invention is directed to a method for modulating (including inhibiting, antagonizing and the like) gamma-secretase, comprising administering an effective amount of a compound of formula I to a patient in need of treatment.

Another embodiment of this invention is directed to a method of treating one or more neurodegenerative diseases, comprising administering an effective amount of one or more (e.g., one) compounds of formula I to a patient in need of treatment.

Another embodiment of this invention is directed to a method of treating one or more neurodegenerative diseases, comprising administering an effective amount of a compound of formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of one or more (e.g., one) compounds of formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of a compound of formula I to a patient in need of treatment.

Another embodiment of this invention is directed to a method of treating Alzheimer's disease, comprising administering an effective amount of one or more (e.g., one) compounds of formula I to a patient in need of treatment.

Another embodiment of this invention is directed to a method of treating Alzheimer's disease, comprising administering an effective amount of a compound of formula I to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula I and the administration of an effective amount of one or more (e.g., one) other pharmaceutical active ingredients (e.g., drugs). The compounds of formula I and the other drugs can be administered separately (i.e., each is in its own separate dosage form), or the compounds of formula I can be combined with the other drugs in the same dosage form.

Thus, other embodiments of this invention are directed to any one of the methods of treatment, or methods of inhibiting, described herein, wherein an effective amount of the compound of formula I is used in combination with an effective amount of one or more other pharmaceutically active ingredients selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe).

Another embodiment of this invention is directed to a method of treating Alzheimer's disease, comprising administering an effective amount of one or more (e.g., one) compounds of formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound of formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of one or more (e.g., one) compounds of formula I, in combination with an effective amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective amount of one or more (e.g., one) compounds of formula I, in combination with an effective amount of one or more BACE inhibitors.

Another embodiment of this invention is directed to a method of treating Alzheimer's disease, comprising administering an effective amount of one or more compounds of formula I, in combination with an effective amount of Exelon (rivastigmine).

Another embodiment of this invention is directed to a method of treating Alzheimer's disease, comprising administering an effective amount of one or more compounds of formula I, in combination with an effective amount of Cognex (tacrine).

Another embodiment of this invention is directed to a method of treating Alzheimer's disease, comprising administering an effective amount of one or more compounds of formula I, in combination with an effective amount of a Tau kinase inhibitor.

Another embodiment of this invention is directed to a method of treating Alzheimer's disease, comprising administering an effective amount of one or more compounds of formula I, in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds of formula I, in combination with an effective amount of one anti-Abeta vaccination (active immunization).

Another embodiment of this invention is directed to a method of treating Alzheimer's disease, comprising administering an effective amount of one or more compounds of formula I, in combination with an effective amount of one or more APP ligands.

Another embodiment of this invention is directed to a method of treating Alzheimer's disease, comprising administering an effective amount of one or more compounds of formula I, in combination with an effective amount of one or more agents that upregulate insulin degrading enzyme and/or neprilysin.

Another embodiment of this invention is directed to a method of treating Alzheimer's disease, comprising administering an effective amount of one or more compounds of formula I, in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of formula I, in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula I, in combination with an effective amount of one or more LXR agonists.

Another embodiment of this invention is directed to a method of treating Alzheimer's disease, comprising administering an effective amount of one or more compounds of formula I, in combination with an effective amount of one or more LRP mimics.

Another embodiment of this invention is directed to a method of treating Alzheimer's disease, comprising administering an effective amount of one or more compounds of formula I, in combination with an effective amount of one or more 5-HT6 receptor antagonists.

Another embodiment of this invention is directed to a method of treating Alzheimer's disease, comprising administering an effective amount of one or more compounds of formula I, in combination with an effective amount of one or more nicotinic receptor agonists.

Another embodiment of this invention is directed to a method of treating Alzheimer's disease, comprising administering an effective amount of one or more compounds of formula I, in combination with an effective amount of one or more H3 receptor antagonists.

This invention also provides a method of treating Alzheimer's disease, comprising administering an effective amount of one or more compounds of formula I, in combination with an effective amount of one or more histone deacetylase inhibitors.

Another embodiment of this invention is directed to a method of treating Alzheimer's disease, comprising administering an effective amount of one or more compounds of formula I, in combination with an effective amount of one or more hsp90 inhibitors.

Another embodiment of this invention is directed to a method of treating Alzheimer's disease, comprising administering an effective amount of one or more compounds of formula I, in combination with an effective amount of one or more m1 muscarinic receptor agonists.

Another embodiment of this invention is directed to a method of treating Alzheimer's disease, comprising administering an effective amount of one or more compounds of formula I, in combination with an effective amount of one or more 5-HT6 receptor antagonists mGluR1 or mGluR5 positive allosteric modulators or agonists Another embodiment of this invention is directed to a method of treating Alzheimer's disease, comprising administering an effective amount of one or more compounds of formula I, in combination with an effective amount of one or more mGluR2/3 antagonists.

Another embodiment of this invention is directed to a method of treating Alzheimer's disease, comprising administering an effective amount of one or more compounds of formula I, in combination with an effective amount of one or more anti-inflammatory agents that can reduce neuroinflammation.

Another embodiment of this invention is directed to a method of treating Alzheimer's disease, comprising administering an effective amount of one or more compounds of formula I, in combination with an effective amount of one or more Prostaglandin EP2 receptor antagonists.

This invention also provides a method of treating Alzheimer's disease, comprising administering an effective amount of one or more compounds of formula I, in combination with an effective amount of one or more PAI-1 inhibitors.

This invention also provides a method of treating Alzheimer's disease, comprising administering an effective amount of one or more compounds of formula I, in combination with an effective amount of one or more agents that can induce Abeta efflux such as gelsolin.

This invention also provides a method of treating Downs syndrome, comprising administering an effective amount of one or more (e.g., one) compounds of formula I to a patient in need of treatment.

This invention also provides a method of treating Downs syndrome, comprising administering an effective amount of a compound of formula I to a patient in need of treatment.

This invention also provides a method of treating Downs syndrome, comprising administering an effective amount of one or more (e.g., one) compounds of formula I, in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective amount of a compound of formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating mild cognitive impairment, comprising administering an effective amount of one or more (e.g., one) compounds of formula I to a patient in need of treatment.

This invention also provides a method of treating glaucoma, comprising administering an effective amount of one or more (e.g., one) compounds of formula I to a patient in need of treatment.

This invention also provides a method of treating cerebral amyloid angiopathy, comprising administering an effective amount of one or more (e.g., one) compounds of formula I to a patient in need of treatment.

This invention also provides a method of treating stroke, comprising administering an effective amount of one or more (e.g., one) compounds of formula I to a patient in need of treatment.

This invention also provides a method of treating dementia, comprising administering an effective amount of one or more (e.g., one) compounds of formula I to a patient in need of treatment.

This invention also provides a method of treating microgliosis, comprising administering an effective amount of one or more (e.g., one) compounds of formula I to a patient in need of treatment.

This invention also provides a method of treating brain inflammation, comprising administering an effective amount of one or more (e.g., one) compounds of formula I to a patient in need of treatment.

This invention also provides a method of treating olfactory function loss, comprising administering an effective amount of one or more (e.g., one) compounds of formula I to a patient in need of treatment.

This invention also provides combinations (i.e., pharmaceutical compositions) comprising an effective amount of one or more (e.g., one) compounds of formula I, in combination with an effective amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of a compound of formula I in a pharmaceutically acceptable carrier, and another container (i.e., a second container) comprises an effective amount of another pharmaceutically active ingredient (as described above), the combined quantities of the compound of formula I and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Another embodiment of this invention is directed to a pharmaceutically acceptable salt of a compound of formula (I), said compound of formula (I) being selected from the group consisting of: 1 to 48.

Another embodiment of this invention is directed to a pharmaceutically acceptable ester of a compound of formula (I), said compound of formula (I) being selected from the group consisting of: 1 to 48.

Another embodiment of this invention is directed to a solvate of a compound of formula (I), said compound of formula (I) being selected from the group consisting of: 1 to 48.

Another embodiment of this invention is directed to a compound of formula (I) in isolated form, said compound of formula (I) being selected from the group consisting of: 1 to 48.

Another embodiment of this invention is directed to a compound of formula (I) in pure form, said compound of formula (I) being selected from the group consisting of: said compound of formula (I) being selected from the group consisting of: 1 to 48.

Another embodiment of this invention is directed to a compound of formula (I) in pure and isolated form, said compound of formula (I) being selected from the group consisting of: said compound of formula (I) being selected from the group consisting of: 1 to 48.

Another embodiment is directed to a pharmaceutical composition comprising an effective amount of one or more (e.g., one) compounds of formula (I) and a pharmaceutically acceptable carrier, said compound of formula (I) being selected from the group consisting of: said compound of formula (I) being selected from the group consisting of: 1 to 48.

Another embodiment is directed to a pharmaceutical composition comprising an effective amount of a pharmaceutically acceptable salt of one or more (e.g., one) compounds of formula (I) and a pharmaceutically acceptable carrier, said compound of formula (I) being selected from the group consisting of: said compound of formula (I) being selected from the group consisting of: 1 to 48.

Another embodiment is directed to a pharmaceutical composition comprising an effective amount of a pharmaceutically acceptable ester of one or more (e.g., one) compounds of formula (I) and a pharmaceutically acceptable carrier, said compound of formula (I) being selected from the group consisting of: said compound of formula (I) being selected from the group consisting of: 1 to 48.

Another embodiment is directed to a pharmaceutical composition comprising an effective amount of a solvate of one or more (e.g., one) compounds of formula (I) and a pharmaceutically acceptable carrier, said compound of formula (I) being selected from the group consisting of: said compound of formula (I) being selected from the group consisting of: 1 to 48.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase, said compound of formula (I) being selected from the group consisting of: said compound of formula (I) being selected from the group consisting of: 1 to 48.

Another embodiment of this invention is directed to a pharmaceutical composition comprising an effective amount of one or more (e.g., one) compounds of formula (I), and effective amount of one or more BACE inhibitors, and a pharmaceutically acceptable carrier, said compound of formula (I) being selected from the group consisting of: said compound of formula (I) being selected from the group consisting of: 1 to 48.

Another embodiment of this invention is directed to a pharmaceutical composition comprising an effective amount of one or more (e.g., one) compounds selected from the group consisting of: 1 to 48, and an effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more muscarinic antagonists (e.g., m₁ agonists or m₂ antagonists), and a pharmaceutically acceptable carrier.

Another embodiment of this invention is directed to a pharmaceutical composition comprising an effective amount of one or more (e.g., one) compounds selected from the group consisting of: 1 to 48, and effective amount of Exelon (rivastigmine), and a pharmaceutically acceptable carrier.

Another embodiment of this invention is directed to a pharmaceutical composition comprising an effective amount of one or more (e.g., one) compounds selected from the group consisting of: 1 to 48, and effective amount of Cognex (tacrine), and a pharmaceutically acceptable carrier.

Another embodiment of this invention is directed to a pharmaceutical composition comprising an effective amount of one or more (e.g., one) compounds selected from the group consisting of: 1 to 48, and effective amount of a Tau kinase inhibitor, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds selected from the group consisting of: 1 to 48, and effective amount of one anti-Abeta vaccine (active immunization), and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more APP ligands, and a pharmaceutically acceptable carrier.

Another embodiment of this invention is directed to a pharmaceutical composition comprising an effective amount of one or more (e.g., one) compounds selected from the group consisting of: consisting of: 1 to 48, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds selected from the group consisting of: consisting of: 1 to 48, and effective amount of one or more fibrates (for example, clofibrate, Clofibride, Etofibrate, Aluminium Clofibrate), and a pharmaceutically acceptable carrier.

Another embodiment of this invention is directed to a pharmaceutical composition comprising an effective amount of one or more (e.g., one) compounds selected from the group consisting of: consisting of: 1 to 48, and effective amount of one or more LXR agonists, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more LRP mimics, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more 5-HT6 receptor antagonists, and a pharmaceutically acceptable carrier.

Another embodiment of this invention is directed to a pharmaceutical composition comprising an effective amount of one or more (e.g., one) compounds selected from the group consisting of: consisting of: 1 to 48, and effective amount of one or more nicotinic receptor agonists, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more H3 receptor antagonists, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more histone deacetylase inhibitors, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more hsp90 inhibitors, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more m1 muscarinic receptor agonists, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more one mGluR2/3 antagonists, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more PAI-1 inhibitors, and a pharmaceutically acceptable carrier.

Another embodiment of this invention is directed to a pharmaceutical composition comprising an effective amount of one or more (e.g., one) compounds selected from the group consisting of: 1 to 48, and effective amount of one or more agents that can induce Abeta efflux such as gelsolin, and a pharmaceutically acceptable carrier.

The compounds of formulas (I) selected from the group consisting of: 1 to 48 can be useful as gamma secretase modulators and can be useful in the treatment and prevention of diseases such as, for example, central nervous system disorders (such as Alzheimers disease and Downs Syndrome), and treating mild cognitive impairment, glaucoma, cerebral amyloid angiopathy, stroke, dementia, microgliosis, brain inflammation, and olfactory function loss.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment, said compound of formula (I) being selected from the group consisting of: 1 to 48.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment, said compound of formula (I) being selected from the group consisting of: 1 to 48.

Another embodiment of this invention is directed to a method of treating one or more neurodegenerative diseases, comprising administering an effective (i.e., therapeutically effective) amount of one or more (e.g., one) compounds of formula (I) to a patient in need of treatment, said compound of formula (I) being selected from the group consisting of: 1 to 48.

Another embodiment of this invention is directed to a method of treating one or more neurodegenerative diseases, comprising administering an effective (i.e., therapeutically effective) amount of a compound of formula (I) to a patient in need of treatment, said compound of formula (I) being selected from the group consisting of: 1 to 48.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment, said compound of formula (I) being selected from the group consisting of: 1 to 48.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment, said compound of formula (I) being selected from the group consisting of: 1 to 48.

Another embodiment of this invention is directed to a method of treating Alzheimer's disease, comprising administering an effective (i.e., therapeutically effective) amount of one or more (e.g., one) compounds of formula (I) to a patient in need of treatment, said compound of formula (I) being selected from the group consisting of: 1 to 48.

Another embodiment of this invention is directed to a method of treating Alzheimer's disease, comprising administering an effective (i.e., therapeutically effective) amount of a compound of formula (I) to a patient in need of treatment, said compound of formula (I) being selected from the group consisting of: 1 to 48.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment, said compound of formula (I) being selected from the group consisting of: 1 to 48.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment, said compound of formula (I) being selected from the group consisting of: 1 to 48.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I), and the administration of one or more (e.g., one) other pharmaceutical active ingredients (e.g., drugs). The compounds of formula (I), and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein the compounds of formula (I) are used in combination with an effective amount of one or more other pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) selected from the group consisting of: 1 to 48, and the administration of one or more (e.g., one) other pharmaceutical active ingredients (e.g., drugs). The compounds of formula I to 48, and the other drugs can be administered separately (i.e., each is in its own separate dosage form), or the compounds of formula 1 to 48 can be combined with the other drugs in the same dosage form.

Thus, other embodiments of this invention are directed to any one of the methods of treatment, or methods of inhibiting, described herein, wherein the compounds of formula (I), selected from the group consisting of: 1 to 48 are used in combination with an effective amount of one or more other pharmaceutically active ingredients selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1, mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Other embodiments of this invention are directed to any one of the methods of treatment, or methods of inhibiting, described herein, wherein the compounds of formula (I), selected from the group consisting of: 1 to 48 are used in combination with an effective amount of one or more other pharmaceutically active ingredients selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; and cholesterol absorption inhibitors (e.g., ezetimibe).

Other embodiments of this invention are directed to any one of the methods of treatment, or methods of inhibiting, described herein, wherein the compounds of formula (I), selected from the group consisting of: 1 to 48 are used in combination with an effective amount of one or more other pharmaceutically active ingredients selected from the group consisting of: Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a method of treating Alzheimer's disease, comprising administering an effective (i.e., therapeutically effective) amount of one or more (e.g., one) compounds of formula (I) selected from the group consisting of: 1 to 48, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of a compound of formula (I) selected from the group consisting of: 1 to 48, in combination with an effective (i.e., therapeutically effective) amount of one or more (e.g., one) cholinesterase inhibitors (such as, for example, (t)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of one or more (e.g., one) compounds of formula (I) selected from the group consisting of: in combination with an effective (i.e., therapeutically effective) amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective (i.e., therapeutically effective) amount of one or more (e.g., one) compounds of formula (I) selected from the group consisting of: 1 to 48, in combination with an effective (i.e., therapeutically effective) amount of one or more BACE inhibitors.

Another embodiment of this invention is directed to a method of treating Alzheimer's disease, comprising administering an effective (i.e., therapeutically effective) amount of a compound of formula (I) selected from the group consisting of: 1 to 48, and the Compound of Example 1, in combination with an effective (i.e., therapeutically effective) amount of one or more BACE inhibitors.

Another embodiment of this invention is directed to a method of treating Downs syndrome, comprising administering an effective (i.e., therapeutically effective) amount of one or more (e.g., one) compounds of formula (I) selected from the group consisting of: 1 to 48 to a patient in need of treatment.

Another embodiment of this invention is directed to a method of treating Downs syndrome, comprising administering an effective (i.e., therapeutically effective) amount of a compound of formula (I) selected from the group consisting of: 1 to 48, to a patient in need of treatment.

Another embodiment of this invention is directed to a method of treating Downs syndrome, comprising administering an effective (i.e., therapeutically effective) amount of one or more (e.g., one) compounds of formula (I) selected from the group consisting of: 1 to 48, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective (i.e., therapeutically effective) amount of a compound of formula (I) selected from the group consisting of: 1 to 48, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to combinations (i.e., pharmaceutical compositions) comprising an effective (i.e., therapeutically effective) amount of one or more (e.g., one) compounds of formula (I) selected from the group consisting of: 1 to 48 in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of one or more (e.g., one) compounds of formula (I) (e.g., compounds selected from the group consisting of: 1 to 48 in a pharmaceutically acceptable carrier, and another container (i.e., a second container) comprises an effective amount of another pharmaceutically active ingredient (as described above), the combined quantities of the compounds of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

This invention also provides a kit comprising, in separate containers, in a single package, pharmaceutical compositions for use in combination, wherein one container comprises an effective amount of a compound selected from the group consisting of the compounds of formulas (I) (e.g. the compounds selected from the group consisting of: 1 to 48) in a pharmaceutically acceptable carrier, and another container (i.e., a second container) comprises an effective amount of another pharmaceutically active ingredient (as described above), the combined quantities of the compound of formulas (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above methods of treatment, pharmaceutical compositions, or kits wherein the compound of formula I is any one of the compounds 1 to 48.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Yet another aspect of this invention is a kit comprising an amount of at least one compound of Formula I, or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative example which should not be construed to limit the scope of the disclosure. Alternative mechanistic pathways and analogous structures will be apparent to those skilled in the art.

### ILLUSTRATIVE EXAMPLES

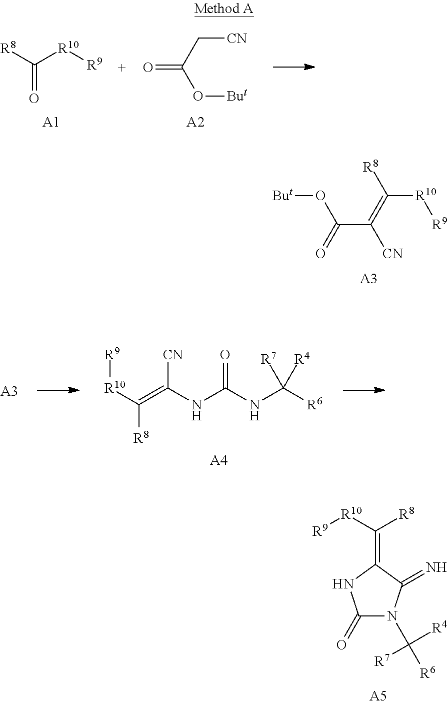

### Method A, Step 1

To a THF solution of A1 (R⁸═H; R¹⁰=3-MeO-phenyl; R⁹=4-(4-Methylimidazol-1-yl)) and A2 (1:1) will be added KOBu^{t} (10%) and the mixture will be stirred until the starting material is consumed. The reaction mixture will be partitioned in DCM/Water and the organic layer will be dried and solvent evaporated to give compound A3 (R⁸═H; R¹⁰=3-MeO-phenyl; R⁹=(4-(4-Methylimidazol-1-yl)) after chromatography purification.

### Method A, Step 2

Compound A3 (R⁸═H; R¹⁰=3-MeO-phenyl; R⁹=4-(4-Methylimidazol-1-yl)) will be treated with 20% TFA in DCM After the starting material is consumed, the volatiles will be evaporated to give the corresponding acid which will be treated with diphenylphosphoryl azide (1 eq) and DBU (1.0 eq) in toluene before the reaction mixture is refluxed for 2 h. To the cooled reaction mixture will be added p-F-α-methylbenzylamine (1.0 eq) and the solvent will be evaporated. The residue will be purified to give compound A4 (R⁸═H; R¹⁰=3-MeO-phenyl; R⁹=4-(4-Methylimidazol-1-yl); R4=Me; R6=H and R7=p-F-phenyl).

### Method A, Step 3

To a DMF solution of A4 (R⁸═H; R¹⁰=3-MeO-phenyl; R⁹=4-(4-Methylimidazol-1-yl); R4=Me; R6=H and R7=p-F-phenyl) will be added NaH (1.1 eq) and the solution will be stirred at a temperature from r.t. to 100° C. until starting material is consumed. The volatiles will be removed and residue purified via reverse phase chromatography to give compound A5 (R⁸═H; R¹⁰=3-MeO-phenyl; R⁹=4-(4-Methylimidazol-1-yl); R4=Me; R6=H and R7=p-F-phenyl).

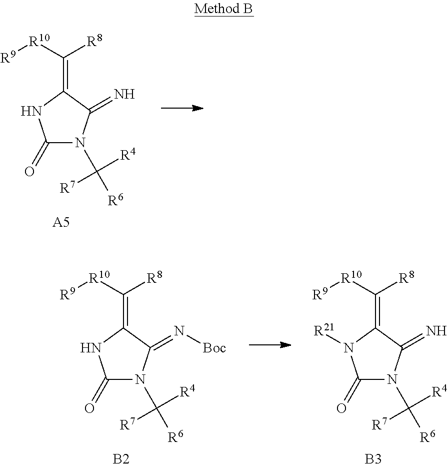

### Method B, Step 1

To a DCM solution of A5 (R⁸═H; R¹⁰=3-MeO-phenyl; R⁹=4-(4-Methylimidazol-1-yl); R4=Me; R6=H and R7=p-F-phenyl) and triethylamine (1.1 eq) will be added BOC₂O (1.1 eq) and the solution stirred until disappearance of starting material before the volatiles are removed and residue purified via chromatography to give compound B2 (R⁸═H; R¹⁰=3-MeO-phenyl; R⁹=4-(4-Methylimidazol-1-yl); R4=Me; R6=H and R7=p-F-phenyl).

### Method B, Step 2

To a DMF solution of B2 (R⁸═H; R¹⁰=3-MeO-phenyl; R⁹=4-(4-Methylimidazol-1-yl); R4=Me; R6=H and R7=p-F-phenyl) will be added R²¹—I (R²¹=Me, 1.1 eq) and NaH (1.1 eq) and the solution will be stirred until starting material disappears. The volatiles will be removed and residue purified to give the product which will be treated with 20% TFA in DCM for 30 min before reverse phase column purification to give B3 (R⁸═H; R¹⁰=3-MeO-phenyl; R⁹=4-(4-Methylimidazol-1-yl); R²¹=ME, R4=Me; R6=H and R7=p-F-phenyl).

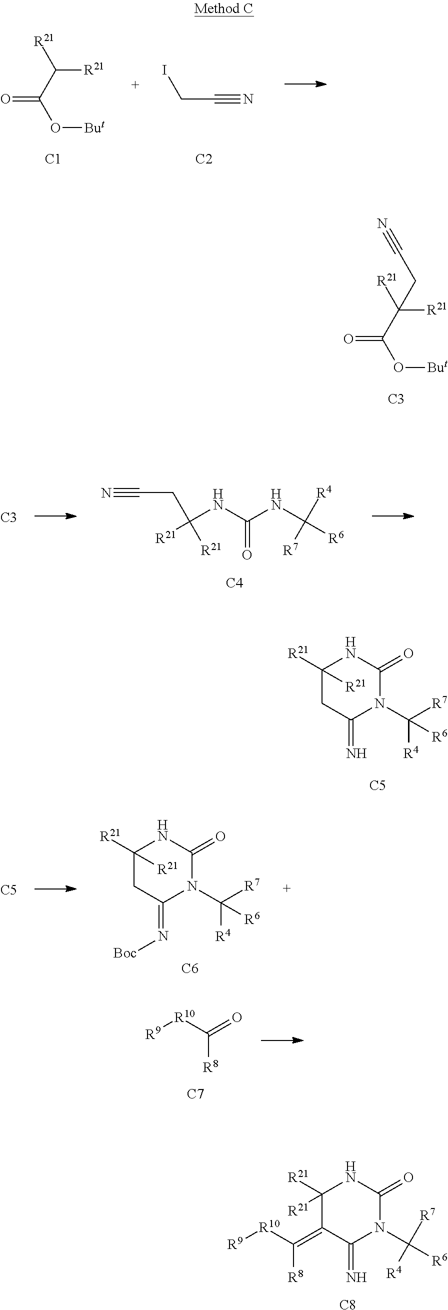

### Method C, Step 1

To a THF solution of C1 (R21=R21=Me) at −78° C. will be added 1.1 eq LDA followed by C2 (1:1 ratio) in THF and the solution will be allowed to warm up to r.t. before the volatiles are removed and residue chromatographed to give compound C3 (R21=R²¹=Me).

### Method C, Step 2

Method similar to Method A step 2 will be used for transformation of C3 (R²¹ ═R²¹=Me) to C4 (R²¹═R²¹=Me and R⁴=Me, R⁶═H and R⁷=p-F-Ph).

### Method C, Step 3

Method similar to Method A step 3 will be used for transformation of C4 (R²¹═R²¹=Me and R⁴=Me, R⁶═H and R⁷=p-F-Ph) to C5 (R²¹═R²¹=Me and R⁴=Me, R⁶═H and R⁷=p-F-Ph).

### Method C, Step 4

Method similar to Method B step 1 will be used for transformation of C5 (R²¹═R²¹=Me and R⁴=Me, R⁶═H and R⁷=p-F-Ph) to C6 (R²¹═R²¹=Me and R⁴=Me, R⁶═H and R⁷=p-F-Ph).

### Method C, Step 5

To a THF solution of C6 (R²¹═R²¹=Me and R⁴=Me, R⁶═H and R⁷=p-F-Ph) will be added LDA (1.1 eq) at −78° C. and the solution will be stirred for 30 min before C7 (R⁸═H; R¹⁰=3-MeO-phenyl; R⁹=4-(4-Methylimidazol-1-yl)) will be added. After the starting material is consumed, the reaction mixture will be treated with triflic anhydride and triethyl amine before the volatiles will be removed and residue chromatographed to give a product which upon TFA deprotection and purification will give compound C8 (R²¹═R²¹=Me and R⁴=Me, R⁶═H and R⁷=p-F-Ph, R⁸═H; R¹⁰=3-MeO-phenyl; R⁹=4-(4-Methylimidazol-1-yl)).

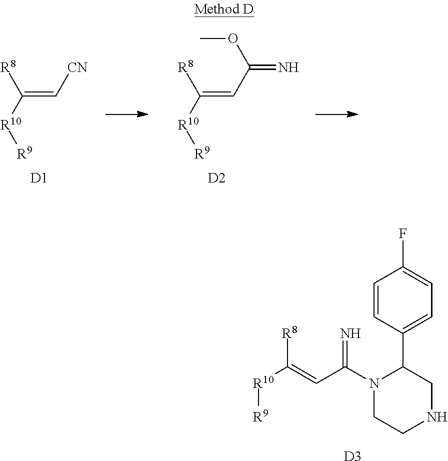

### Method D, Step 1

To a solution of D1 (R⁸═H; R¹⁰=3-MeO-phenyl; R⁹=4-(4-Methylimidazol-1-yl)) in absolute MeOH will be bubbled anhydrous HCl and the final solution will be let stand to form compound D2 (R⁸═H; R¹⁰=3-MeO-phenyl; R⁹=(4-(4-Methylimidazol-1-yl)).

### Method D, Step 2

A DMF solution of 5-Boc-2-p-F-phenyl-2-piperazine and D2 (R⁸═H; R¹⁰=3-MeO-phenyl; R⁹=4-(4-Methylimidazol-1-yl)) will be heated to until the starting material disappears. The product will be purified and boc removed using TFA/DCM to give compound D3 after reverse phase column purification (R⁸═H; R¹⁰=3-MeO-phenyl; R⁹=4-(4-Methylimidazol-1-yl)).

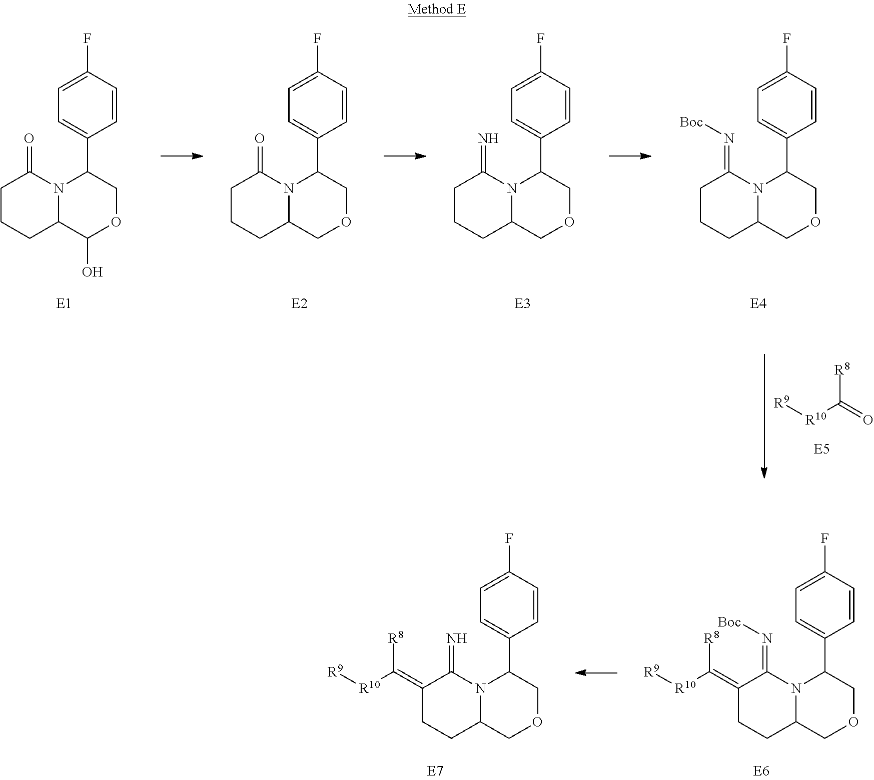

### Method E, Step 1

E1 will be synthesized using a literature procedure: Francois, David; Poupon, Erwan; Kunesch, Nicole; Husson, Henri-Philippe European Journal of Organic Chemistry 2004, (23), 4823-4829.

To a DCM solution of E1 at 0° C. will be added TFA in presence of Triethylsilane. The final product will be purified after the volatiles are removed to give E2

### Method E, Step 2

A literature procedure will be adapted: Bredereck, Hellmut; Bredereck, Karl. Chemische Berichte 1961, 94 2278-95.

Compound E2 will be refluxed with POCl₃. After removal of volatiles, the residue will be treated with NH₃/MeOH to give E3 after purification.

### Method E, Step 3

Compound E3 will be treated with BOC₂O (1.1 eq) in DCM with triethylamine (1.1 eq) to give compound E4 after purification.

### Method E, Step 4

Compound E4 will be dissolved in anhydrous THF at −78° C. and 1 eq of LDA will be added. After 30 min, compound E5 (R⁸═H; R¹⁹=3-MeO-phenyl; R⁹=(4-(4-Methylimidazol-1-yl)) will be added. The reaction will be allowed to warm up to r.t. and treated with triflic anhydride (1.1 eq) and triethyl amine before the volatiles will be removed. The residue will be purified to give compound E6 (R⁸═H; R¹⁹=3-MeO-phenyl; R⁹=4-(4-Methylimidazol-1-yl)).

### Method E, Step 5

Compound E6 (R⁸═H; R¹⁹=3-MeO-phenyl; R⁹=4-(4-Methylimidazol-1-yl)) will be treated with 20% TFA/DCM for 30 min before the volatiles are removed and residue purified to give E7(R⁸═H; R¹⁰=3-MeO-phenyl; R⁹=(4-(4-Methylimidazol-1-yl)).

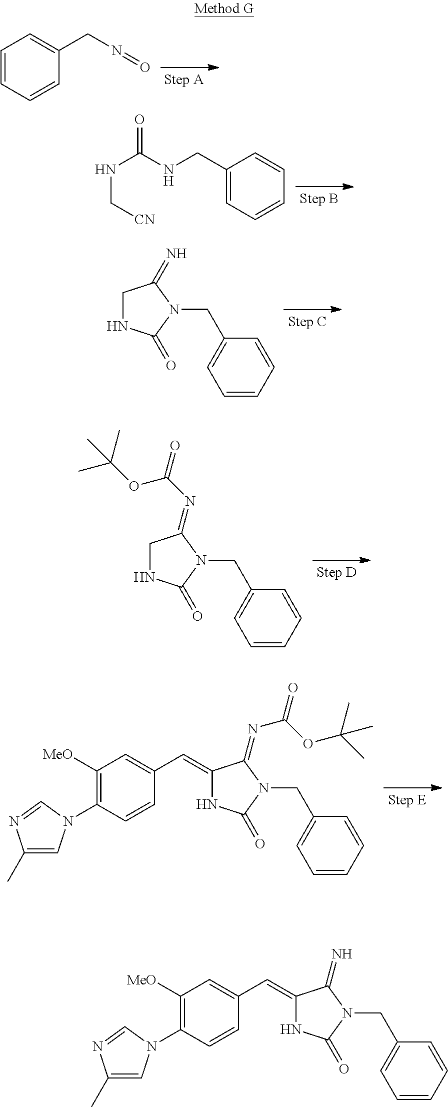

### Method G Step A: 1-Benzyl-3-(cyanomethyl)urea

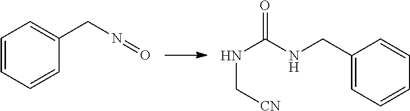

Into a Biotage microwave vial was added aminoacetonitrile hydrogen sulfate (506 mg, 3.28 mmol), methylene chloride (10 mL, 200 mmol), N,N-diisopropylethylamine (0.62 mL, 3.6 mmol), and benzyl isocyanate (0.398 mL, 3.24 mmol). The reaction was stirred at room temperature for 16 hours. The reaction was diluted with brine and EtOAc. The aqueous layer was removed and the organic phase was washed with saturated NH₄Cl (2×). The combined organics were dried (Na₂SO₄), filtered and concentrated to yield 1-benzyl-3-(cyanomethyl)urea (600 mg; Yield=90%).

### Method G, Step B: 1-Benzyl-5-iminoimidazolidin-2-one

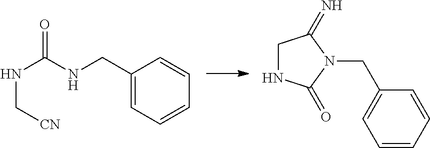

Into a Biotage microwave vial was added sodium hydride (60:40, sodium hydride:mineral oil, 22 mg) and THF (0.5 mL). To this mixture was added 1-benzyl-3-(cyanomethyl)urea (62 mg, 0.33 mmol) as a solution in THF (0.5 mL) and the resulting reaction was stirred at room temperature for 25 minutes. The reaction was slowly quenched with water and diluted with EtOAc. The organic layer was removed and the aqueous phase was extracted with EtOAc (2×). The combined organics were dried (Na₂SO₄), filtered, and concentrated to yield 1-benzyl-5-iminoimidazolidin-2-one (62 mg; Yield=95%).

### Method G, Step C: (E)-tert-Butyl 3-benzyl-2-oxoimidazolidin-4-ylidenecarbamate

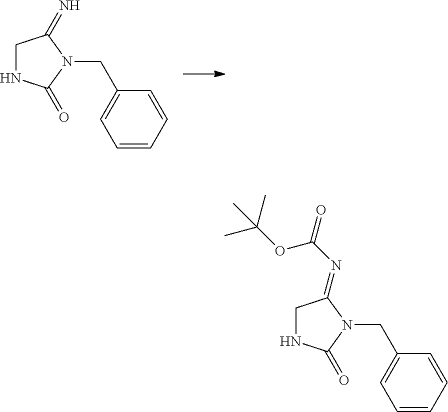

Into a vial was added crude 1-benzyl-5-iminoimidazolidin-2-one (62 mg, 0.31 mmol), di-tert-butyldicarbonate (93 mg, 0.43 mmol), and methylene chloride (1 mL, 20 mmol). The reaction was stirred at room temperature for 16 hours. The reaction was concentrated and the residue was purified by flash chromatography (0% to 100% (15 min) EtOAc/hexanes) to yield (E)-tert-butyl 3-benzyl-2-oxoimidazolidin-4-ylidenecarbamate (41 mg; Yield=46%)

### Method G, Step D: tert-Butyl 3-benzyl-5-(3-methoxy-4-(4-methyl-1H-imidazol-1-yl)benzylidene)-2-oxoimidazolidin-4-ylidenecarbamate

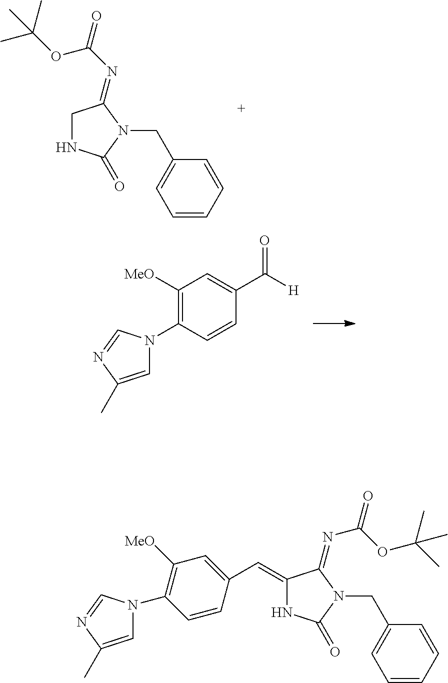

Piperidine (0.0308 mL, 0.312 mmol) was added to an ethanol (20 ml) solution of 3-methoxy-4-(4-methyl-1H-imidazol-1-yl)benzaldehyde (34 mg, 0.16 mmol) and (E)-tert-butyl 3-benzyl-2-oxoimidazolidin-4-ylidenecarbamate (41 mg, 0.14 mmol) and was heated to reflux overnight. The reaction was cooled to room temperature and concentrated. The residue was purified by reverse phase chromatography (10:90 to 100:00 (10 min) CH₃CN/H₂O (0.1% formic acid)) to yield tert-butyl 3-benzyl-5-(3-methoxy-4-(4-methyl-1H-imidazol-1-yl)benzylidene)-2-oxoimidazolidin-4-ylidenecarbamate (32 mg; Yield=39%)

### Method G, Step E: (Z)-1-Benzyl-5-imino-4-(3-methoxy-4-(4-methyl-1H-imidazol-1-yl)benzylidene)imidazolidin-2-one

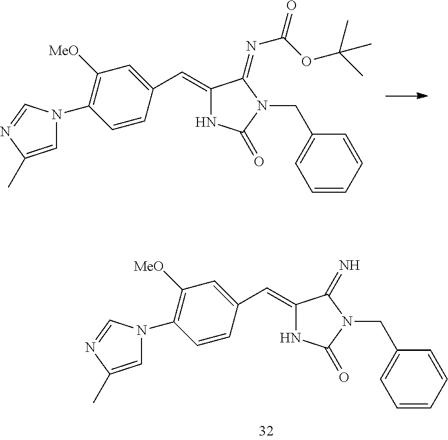

Into a vial was added (Z)-tert-butyl((Z)-3-benzyl-5-(3-methoxy-4-(4-methyl-1H-imidazol-1-yl)benzylidene)-2-oxoimidazolidin-4-ylidene)carbamate (15 mg, 0.031 mmol) and 1 M of trifluoroacetic acid in methylene chloride (1 mL) The reaction was stirred for 1 hour at room temperature. The residue was purified by reverse phase chromatography (10:90 to 100:00 (10 min) CH₃CN/H₂O (0.1% TFA)) to yield (Z)-1-benzyl-5-imino-4-(3-methoxy-4-(4-methyl-1H-imidazol-1-yl)benzylidene)imidazolidin-2-one (7 mg; Yield=60%); NMR (400 MHz, CD₃OD) δ 7.68-7.66 (m, 1H), 7.61 (s, 1H), 7.45-7.37 (m, 7H), 7.28 (s, 1H), 5.07 (s, 2H), 4.01 (s, 3H), 2.44 (s, 3H). LCMS m/e: 388.2 (M+H), Rt=1.93 min. The LCMS Retention Time (min) conditions were: Phenomenex C18 reverse phase, 10% CH₃CH/90% H₂O/0.05% TFA→95% CH₃CN/5% H₂O/0.05% TFA.

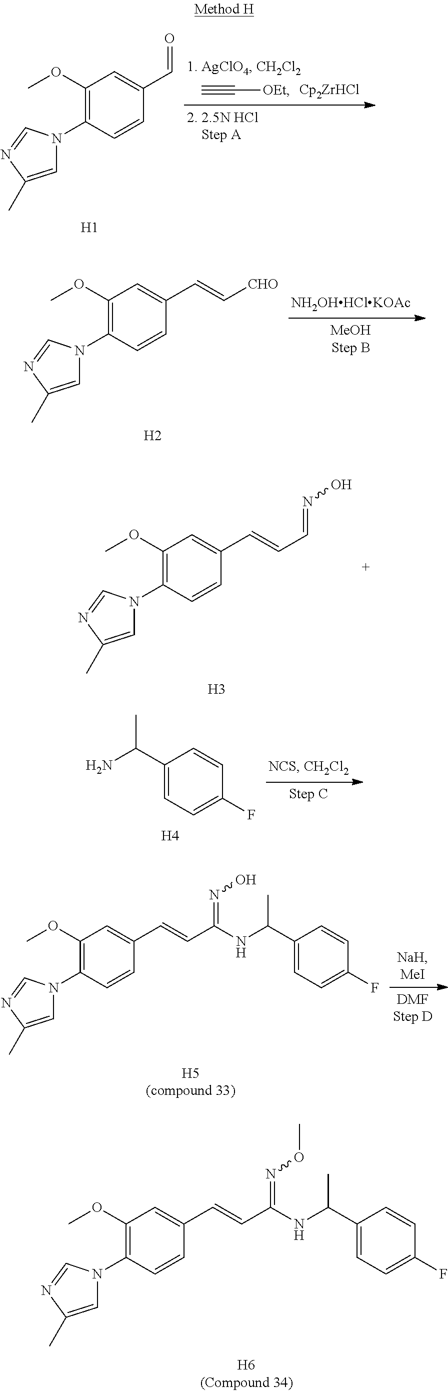

### Method H, Step A

To a suspension of Cp₂ZrHCl (9.02 g, 35.0 mmol) in CH₂Cl₂ (36 ml) was added ethoxyacetylene (50% in hexane, 7.42 ml, 35.0 mmol) at 0° C. under N₂ and stirred at room temperature until it became red clear solution (ca. 10 min). Then H1 (2.16 g, 10.0 mmol) and AgClO₄ (207 mg, 1.0 mmol) were added into the above solution successively and stirred overnight. It was then quenched with saturated aqueous NaHCO₃, stirred for 10 min, filtered through celite to remove solid. The filtrate was partitioned and the aqueous layer was extracted with CH₂Cl₂ (3 times). The combination of organic layers was then concentrated to ca. 30 ml and added with 25 ml 2.5 N HCl and stirred for 1 h. Then it was basified with saturated aqueous NaHCO₃ to PH=8 and partitioned. The aqueous layer was extracted with CH₂Cl₂ (3 times). The combination of organic layers was dried (Na₂SO₄), concentrated to give a crude residue, which was then purified by silica gel flash chromatography (EtOAc) to afford compound H2 as light brown solid (1.85 g, 76%).

### Method H, Step B

To a solution of H2 (530 mg, 2.19 mmol) in MeOH (5 ml) was added NH₂OH.HCl (227 mg, 3.29 mml) and KOAc (322 mg, 3.29 mmol) and stirred at room temperature for 3 h. Then it was filtered to remove solid and the filtrate was concentrated to give a crude residue, which was then purified by silica gel flash chromatography (CH₂Cl₂:MeOH) to afford compound H3 as brown oil (500 mg, 89%).

### Method H, Step C

To a solution of H3 (140 mg, 0.55 mmol) in CH₂Cl₂ (1.2 ml) was added NCS (75 mg, 0.60 mmol) and stirred at 0° C. for 1 h. Then H4 (125 mg, 0.90 mmol) and Et₃N (0.15 ml, 1.09 mmol) were added to the above solution and stirred overnight. It was quenched with saturated aqueous NH₄Cl, extracted with CH₂Cl₂ (3 times). The combination of organic layers was dried (Na₂SO₄), concentrated to give a crude residue, which was then purified by silica gel flash chromatography (CH₂Cl₂:MeOH) to afford compound H5 as colorless oil (40 mg, 19%). ¹H NMR (CDCl₃, ppm): δ 7.76 (s, 1H), 7.38-7.25 (m, 3H), 7.14 (s, 1H), 7.07-7.00 (m, 4H), 6.94 (d, 1H), 6.52 (d, 1H), 4.80-4.74 (m, 1H), 3.85 (s, 3H), 2.22 (s, 3H), 1.50 (d, 3H); MS (ES-LCMS, M+1) 395.2. Retention Time (min, LCMS): 2.13

### Method H, Step D

To a solution of H5 (30 mg, 0.076 mmol) in DMF (2 ml) was added NaH (3.4 mg, 0.084 mmol) and MeI (5.2 ul, 0.084 mmol) and stirred overnight. Then it was quenched with saturated aqueous NH₄Cl, extracted with CH₂Cl₂ (3 times). The combination of organic layers was dried (Na₂SO₄), concentrated to give a crude residue, which was then purified by silica gel flash chromatography (CH₂Cl₂:MeOH) to afford compound H6 as off-white oil (10 mg, 32%). ¹H NMR (CDCl₃, ppm): δ 7.81 (s, 1H), 7.37-7.26 (m, 3H), 7.16 (s, 1H), 7.10-6.94 (m, 5H), 6.53 (d, 1H), 4.82-4.72 (m, 1H), 3.86 (s, 3H), 3.82 (s, 3H), 2.22 (s, 3H), 1.48 (d, 3H); MS (ES-LCMS, M+1) 409.2. Retention Time (min, LCMS): 2.43.

The compounds in Table 2 were prepared by Method G.

TABLE 2

| | | Retention |
|---|---|---|
| | | Time |
| | | (min, |
| Compound Structure | MW (M + 1) | LCMS) |
| 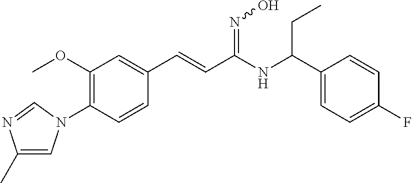 | 409.2 | 2.43 |
| 35 | | |
| 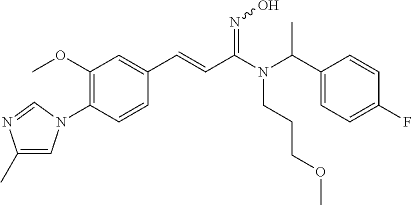 | 467.3 | 2.21 |
| 36 | | |
| 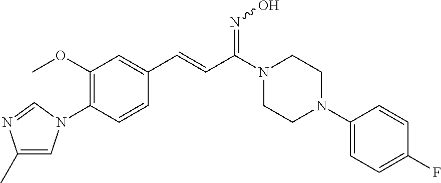 | 436.2 | 2.21 |
| 37 | | |
| 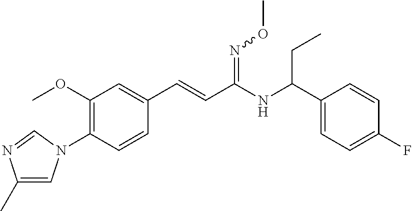 | 423.2 | 2.76 |
| 38 | | |
| 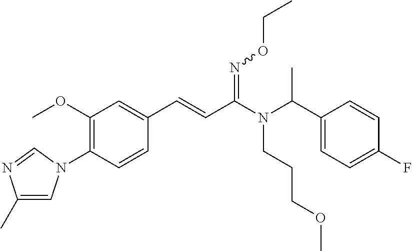 | 495.3 | 3.19 |
| 39 | | |
| 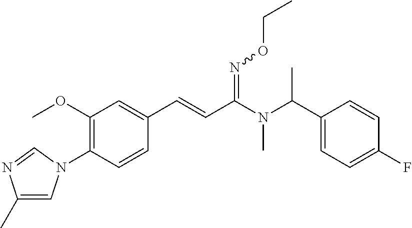 | 437.2 | 3.03 |
| 40 | | |

For Compounds 33 to 40, the LCMS Retention Time (min) conditions were: Phenomenex C18 reverse phase, 10% CH₃CH/90% H₂O/0.05% TFA→95% CH₃CN/5% H₂O/0.05% TFA.

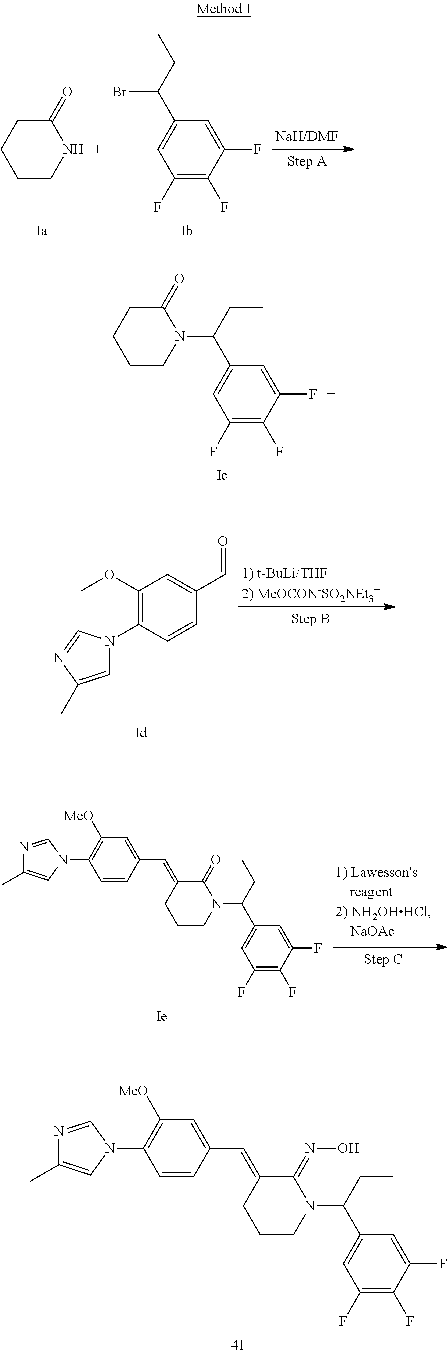

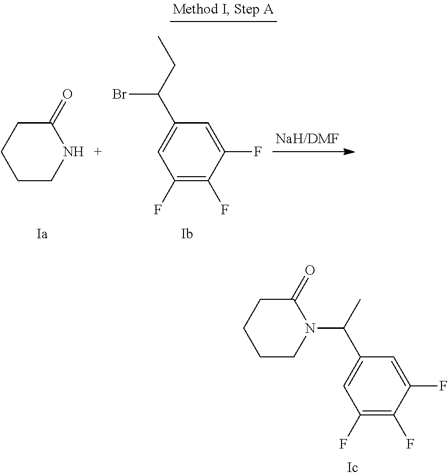

Delta-lactam Ia (1.7 g) was treated with NaH (0.754 g, 60%, 1.1 eq) in DMF (20 mL) at room temperature. The mixture was stirred for 45 minutes before bromide Ib (5.16 g, 1.2 eq) was added dropwise. The reaction mixture was stirred at room temperature overnight. The mixture was diluted with ethyl acetate (300 mL) and NH₄Cl solution (50 mL). The organic layer was washed with water, brine, dried over MgSO₄, and concentrated to give the crude product which was purified by column chromatography eluting with EtOAc/hexanes to yield compound Ic (3.1 g).

### Method I, Step B

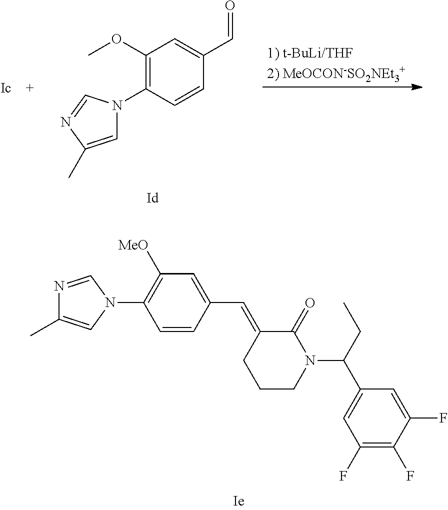

To a solution of compound Ic (1.5 g, 5.53 mmol) in THF was added t-BuLi (3.84 mL, 1.18 eq, 1.7 M) dropwise at −78° C. The mixture was stirred for 45 minutes before aldehyde Id (1.24 g, 1.05 eq) was added in THF (12 mL) at −78° C. The reaction mixture was stirred for 2 hours before it was diluted with ethyl acetate (300 mL) and NH₄Cl solution (50 mL). The organic layer was washed with water, brine, dried over MgSO₄, and concentrated to give the crude product which was taken up in THF and treated with MeOCON⁻SO₂NEt₃⁺ (1.4 eq). The mixture was heated at 80° C. for 2 hours before it was cooled to room temperature. The mixture was directly purified by column chromatography eluting with CH₂Cl₂/MeOH to yield compound Ie.

### Method I, Step C

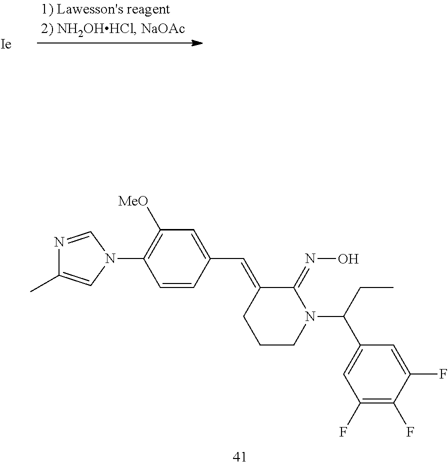

Compound Ie (1 eq) will be treated with Lawesson's regent (1.5 eq) in dioxane and will be heated at 88° C. for 3 hours. Solvent will be removed and the residue will be past through a short pad of celite eluted with CH₂Cl₂/MeOH (v/v 20/1). Solvent will be removed and the residue will be taken up in EtOH. The solution will be treated with NH₂OH.HCl (1.5 eq.) and NaOAc (2.0 eq) and will be stirred at room temperature over night before it will be diluted with EtOAc and NaHCO₃ solution. The organic layer will be washed with water, brine, will be dried over MgSO₄, and will be concentrated which will give the crude product which will be purified by column chromatography eluting with CH₂Cl₂/MeOH which will yield compound 41.

Compound 42:

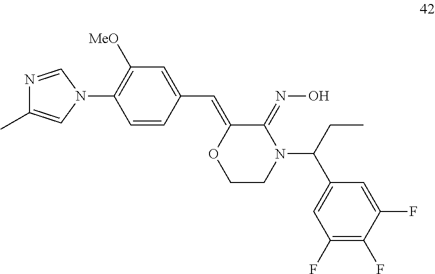

could be prepared by Method I starting with morpholin-3-one.

The following compounds could be prepared by procedures similar to those of Method I:

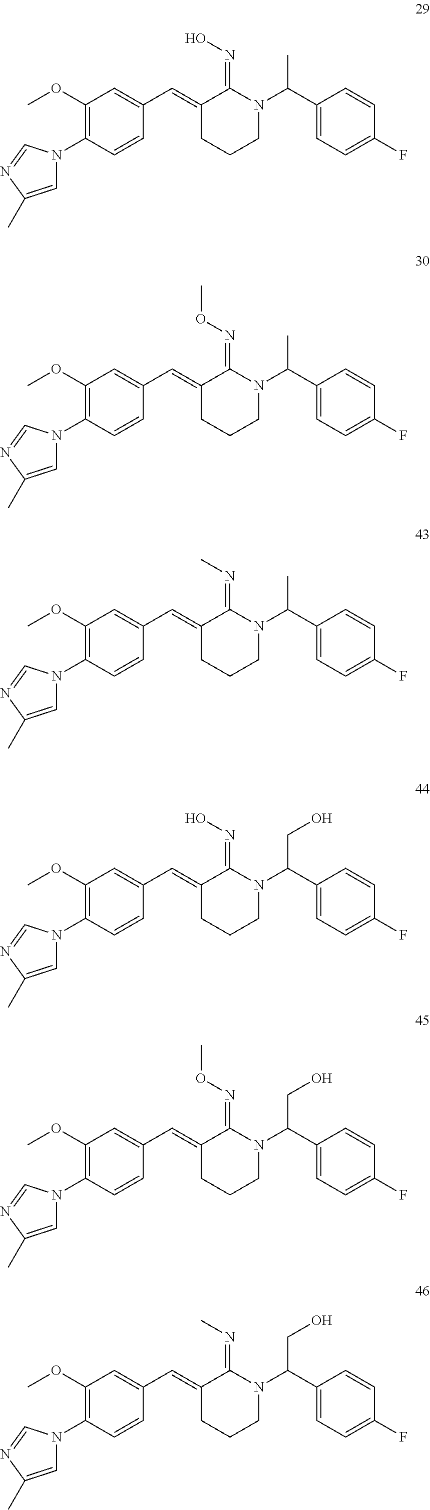

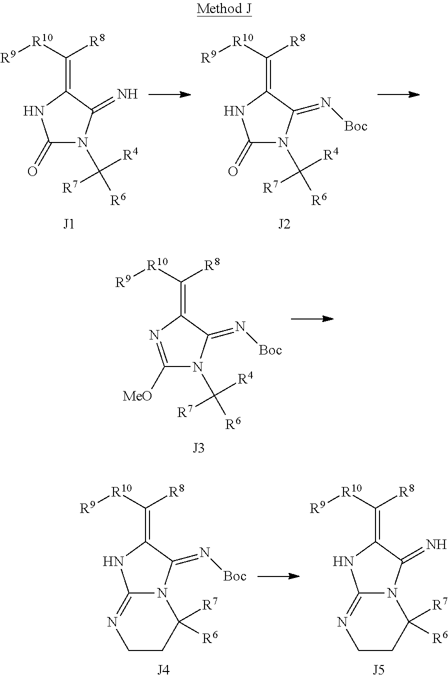

### Method J, Step 1

A method similar to Method B step 1 will be used for transformation of J1 (R⁸═H; R¹⁰=3-MeO-phenyl; R⁹=4-(4-Methylimidazol-1-yl); R4=azidoethyl; R6=Me and R7=p-F-phenyl) to J2 (R⁸═H; R¹⁰=3-MeO-phenyl; R⁹=4-(4-Methylimidazol-1-yl); R4=azidoethyl; R6=Me and R7=p-F-phenyl).

### Method J, Step 2

To a solution of J2 in CH₂Cl₂ will be added methyl trifluoromethanesulfonate (1.1 eq) and the mixture will be stirred until the starting material is consumed. The reaction mixture will be concentrated and the residual crude product J3 (R⁸═H; R¹⁰=3-MeO-phenyl; R⁹=4-(4-Methylimidazol-1-yl); R4=azidoethyl; R6=Me and R7=p-F-phenyl) will be used in the next step without purification.

### Method J, Step 3 and Step 4

To a solution of crude product J3 (R⁸═H; R¹⁰=3-MeO-phenyl; R⁹=4-(4-Methylimidazol-1-yl); R4=azidoethyl; R6=Me and R7=p-F-phenyl) in THF/H₂O (10:1) will be added Ph₃P (2 eq) and the reaction mixture will be stirred overnight. The mixture will be filtered and the filtrate will be concentrated to give a crude residue, which will be treated with 20% TFA in CH₂Cl₂ for 30 min before reverse phase chromatography which will yield compound 47, i.e., compound J5 wherein R⁸═H; R¹⁰=3-MeO-phenyl; R⁹=4-(4-Methylimidazol-1-yl); R6=Me and R7=p-F-phenyl.

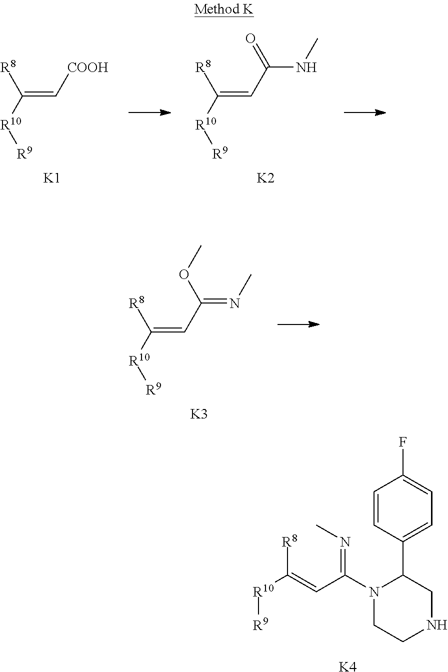

### Method K, Step 1

To a solution of K1 (R⁸═H; R¹⁰=3-MeO-phenyl; R⁹=4-(4-Methylimidazol-1-yl) in THF will be added DCC (1.1 eq) and MeNH₂ (1.1 eq). The mixture will be stirred overnight before the mixture will be filtered to remove the solid. The filtrate will be concentrated and the residue purified to give K2 (R⁸═H; R¹⁰=3-MeO-phenyl; R⁹=4-(4-Methylimidazol-1-yl).

### Method K, Step 2

To a solution of K2 (R⁸═H; R¹⁰=3-MeO-phenyl; R⁹=4-(4-Methylimidazol-1-yl) in CH₂Cl₂ will be added methyl trifluoromethanesulfonate (1.1 eq) and the mixture will be stirred until the starting material is consumed. The mixture will be concentrated and the residual crude product K3 (R⁸═H; R¹⁰=3-MeO-phenyl; R⁹=4-(4-Methylimidazol-1-yl) will be used in the next step without purification.

### Method K, Step 3

A DMF solution of 5-Boc-2-p-F-phenyl-2-piperazine and K3 (R⁸═H; R¹⁰=3-MeO-phenyl; R⁹=4-(4-Methylimidazol-1-yl) will be heated until the starting material disappears. The product will be purified and boc will be removed using TFA/DCM which will give compound 48 after reverse phase column purification, wherein compound 48 is compound K4 wherein R⁸═H; R¹⁰=3-MeO-phenyl; and R⁹=4-(4-Methylimidazol-1-yl).

Compounds 1 to 28, and 31:

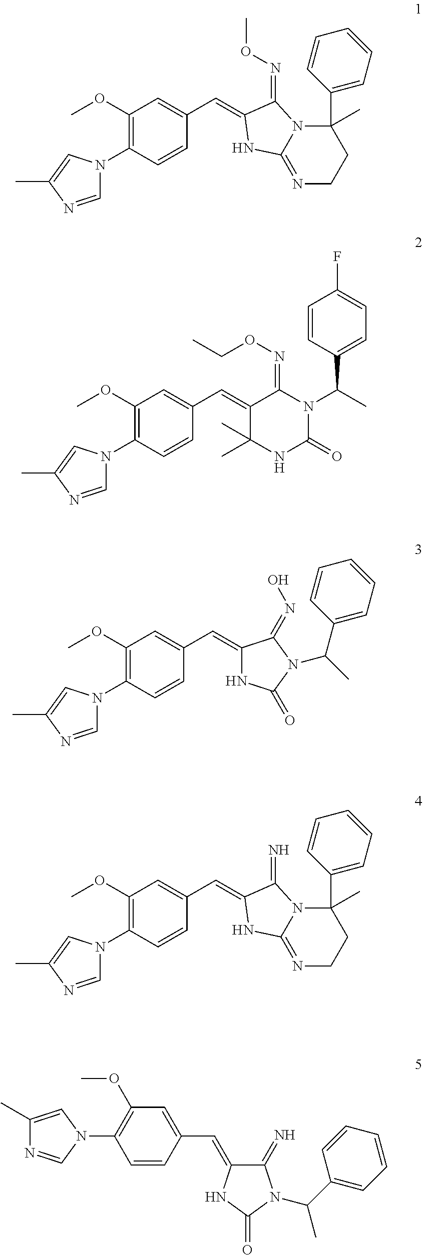

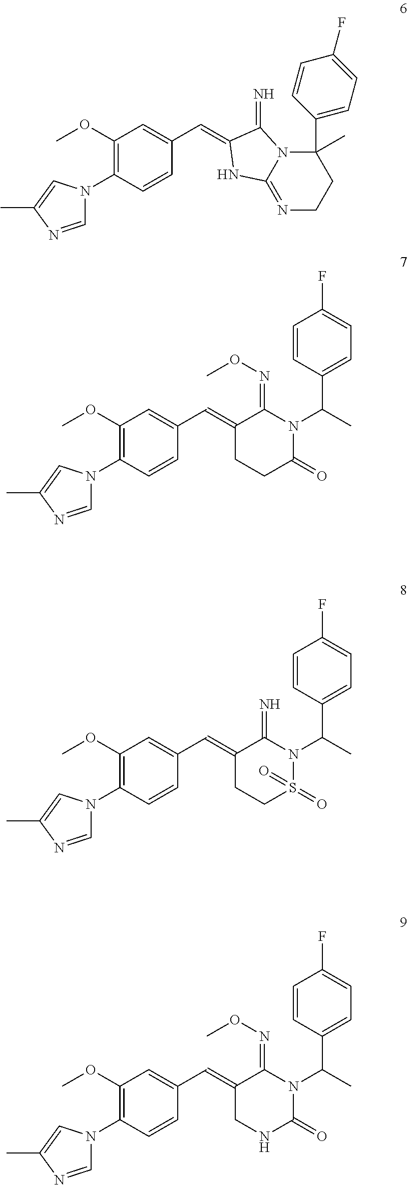

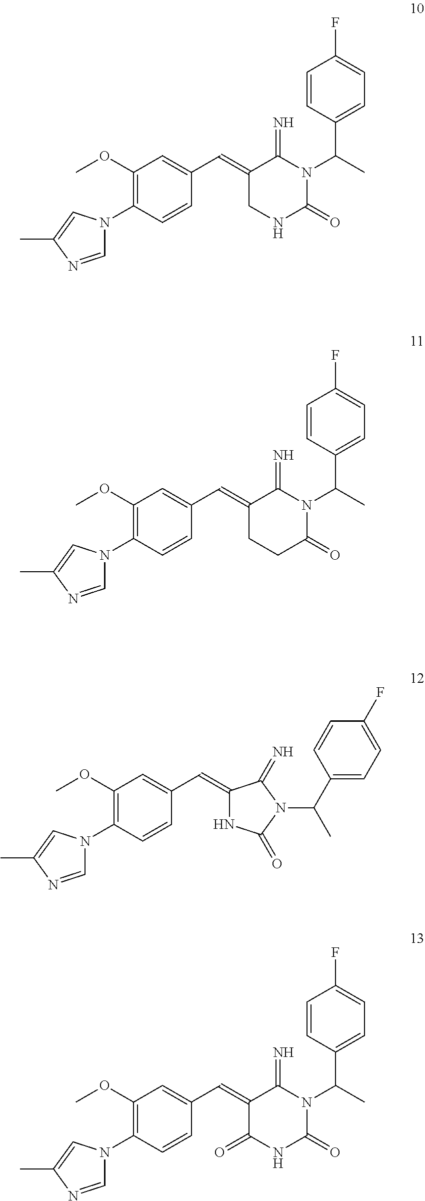

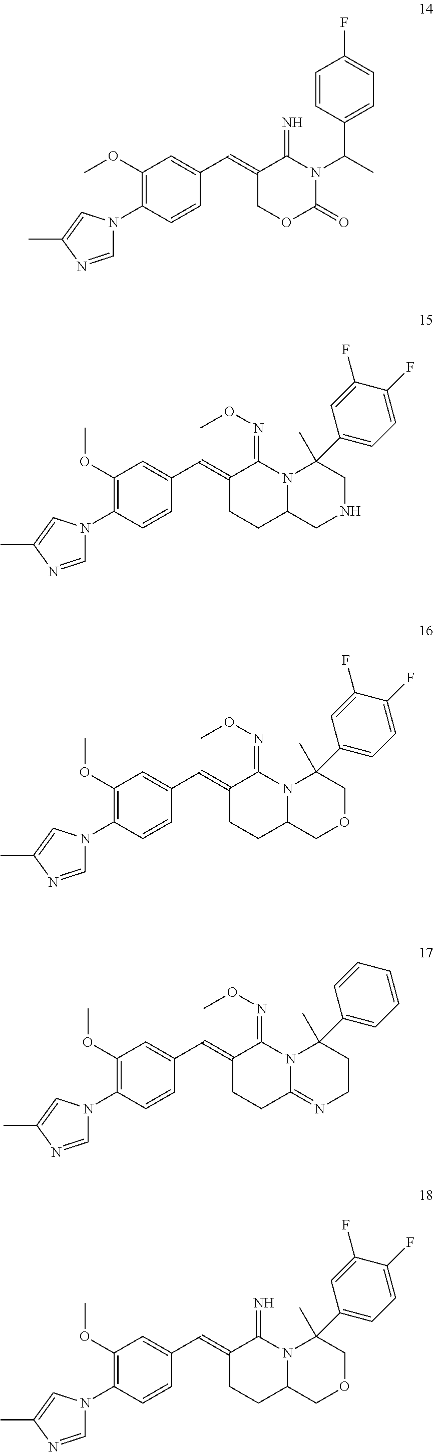

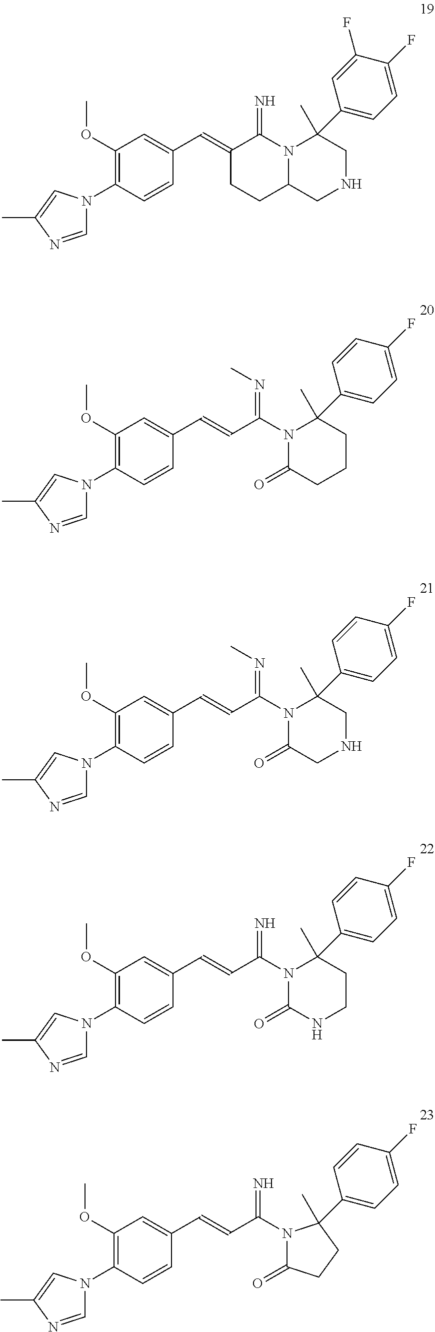

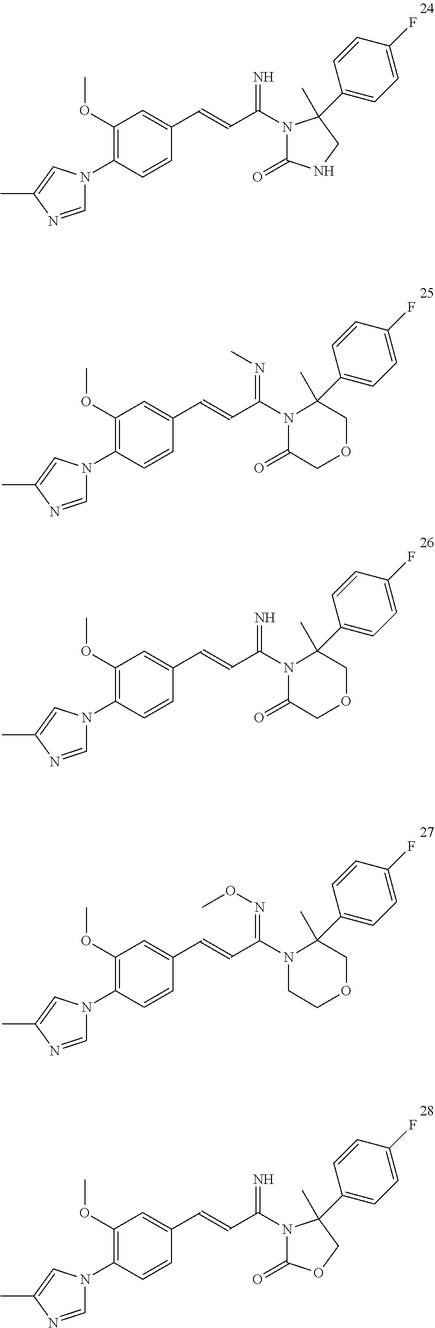

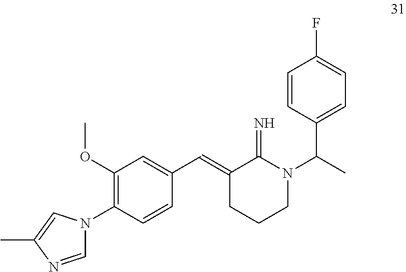

will be synthesized using methods similar to the above methods, as indicated in Table 3 below, and additional adaptation of methods known in the literature:

TABLE 3

| Method | Compounds |
|---|---|
| A | 1 |
| D | 22, 23, 24, 26, and 28 |
| E | 15, 16, 18, and 19 |
| G | 5, 6, 8, 10, 11, 12, 13, and 14 |
| H | 27 |
| I | 2, 3, 7, 9, and 31 |
| J | 1, 4, and 17 |
| K | 20, 21 and 25 |

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-WO2 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody WO2 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated A□ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

Compounds 32 to 40 had a Cellular Ab42 IC50 in the range of 44 nM to 20,000 nM.

While the present invention has been described with in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound of Formula I: 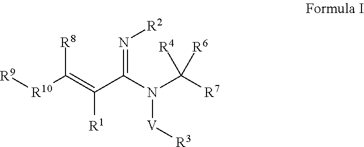
or a pharmaceutically acceptable salt thereof, wherein:
optionally, a ring moiety is formed when either:
(i) R¹ and R³ are joined together to form:
(a) a 5-8 membered heterocyclyl moiety that is optionally substituted with 1-5 independently selected R²¹ groups, or
(b) a 5-8 membered heterocyclyl moiety that is optionally fused with an aryl ring or a heteroaryl ring wherein the ring moiety resulting from the fusion is optionally substituted with 1-5 independently selected R²¹ groups, or
(c) a 5-8 membered heterocyclenyl moiety that is optionally substituted with 1-5 independently selected R²¹ groups, or
(d) a 5-8 membered heterocyclenyl moiety that is optionally fused with an aryl ring or a heteroaryl ring wherein the ring moiety resulting from the fusion is optionally substituted with 1-5 independently selected R²¹ groups; or
(ii) R³ and R⁴ are joined together to form:
a 5-8 membered heterocyclyl moiety that is optionally substituted with 1-5 independently selected R²¹ groups, or
(b) a 5-8 membered heterocyclyl moiety that is optionally fused with an aryl ring or a heteroaryl ring wherein the ring moiety resulting from the fusion is optionally substituted with 1-5 independently selected R²¹ groups, or
(c) a 5-8 membered heterocyclenyl moiety that is optionally substituted with 1-5 independently selected R²¹ groups, or
(d) a 5-8 membered heterocyclenyl moiety that is optionally fused with an aryl ring or a heteroaryl ring wherein the ring moiety resulting from the fusion is optionally substituted with 1-5 independently selected R²¹ groups; or
(iii)
(a) R¹ and R³ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and R³ and R⁴ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, or
(b) R¹ and R³ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and R³ and R⁴ are joined together to form: (1) a 5-8 membered heterocyclyl moiety that is optionally substituted with 1-5 independently selected R²¹ groups, or (2) a 5-8 membered heterocyclenyl moiety that is optionally substituted with 1-5 independently selected R²¹ groups, and wherein the ring moiety resulting from R¹ and R³ being taken together and R³ and R⁴ being taken together is optionally fused with an aryl or heteroaryl ring wherein the ring moiety resulting from the fusion is optionally substituted with 1-5 independently selected R²¹ groups;
and
V is selected from the group consisting of a bond, —O—, —S(O₂)—, —S(O)—, —C(O)—, and —N(R¹⁴)—;
R¹ (when R¹ is not joined to R³), is selected from the group consisting of H, alkyl-, alkenyl- and alkynyl-, aryl-, arylalkyl-, alkylaryl cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocycyl- and heterocyclyalkyl- is optionally substituted with 1-5 independently selected R²¹ substituents;
or, alternatively, R¹ (when not joined with R³) and R⁸ are taken together to form a bond;
R² is independently selected from the group consisting of H, alkyl, cycloalkyl, cycloalkenyl, cycloalkylalky-, heterocycloalkyl, heterocycloalkylalkyl-, aryl, arylalkyl-, heteroaryl, heteroarylalkyl-, —NHR¹⁵, —NR¹⁵R¹⁶, —CN, —OR¹⁵, —C(O)R¹⁵, —C(O)OR¹⁵, —C(O)N(R¹⁵)(R¹⁶), —S(O)₂N(R¹⁵)(R¹⁶), —C(═NOR¹⁵)R¹⁶, —P(O)(OR¹⁵)(OR¹⁶), —S(O)R¹⁵ and —S(O)₂R¹⁵, wherein each of said alkyl, cycloalkyl, cycloalkenyl, cycloalkylalky-, heterocycloalkyl, heterocycloalkylalkyl-, aryl, arylalkyl-, heteroaryl, and heteroarylalkyl- is optionally substituted with 1-5 independently selected R²¹ substituents;
R³ (when R³ is not joined to R¹ or R⁴), is independently selected from the group consisting of H, alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-5 independently selected R²¹ substituents;
R⁴, R⁶ and R⁷ are each independently selected from the group consisting of H, alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-5 independently selected R²¹ substituents;
R⁸ is selected from the group consisting of H, alkenyl- and alkynyl-, aryl-, arylalkyl-, alkylaryl-, cycloalkyl-, cycloalkenyl, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-3 independently selected R²¹ substituents;
R⁹ is selected from the group consisting of alkyl-, alkenyl-, alkynyl-, aryl arylalkyl-, alkylaryl-, cycloalkyl-, cycloalkenyl, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkenyl, cycloalkylalkyl-, heteroaryl-, heteroarylalkyl-, heterocyclyl- and heterocyclyalkyl- is optionally substituted with 1-3 independently selected R²¹ substituents,
R¹⁰ is selected from the group consisting of a bond, alkyl-, alkenyl- and alkynyl-, aryl-, arylalkyl-, alkylaryl-, cycloalkyl-, cycloalkylalkyl-, heteroaryl-, heteroarylalkyl-, heterocyclyl-, heterocyclyalkyl-, 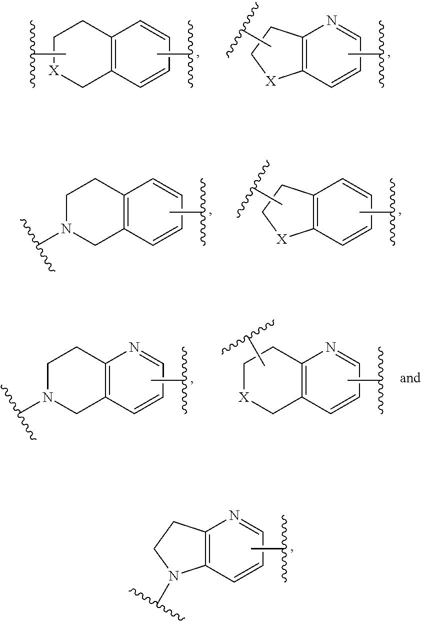
where X is O, N(R¹⁴) or S;
wherein each of said R¹⁰ groups is optionally substituted with 1-3 independently R²¹ substituents;
R¹⁴ is selected from the group consisting of H, alkyl, alkenyl, alkynyl, cycloalkyl, cycloalkenyl, cycloalkylalkyl, cycloalkenyl, heterocyclcyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶); wherein each of said alkyl, alkenyl, alkynyl, cycloalkyl, cycloalkenyl, cycloalkylalkyl, cycloalkenyl, heterocyclyl, heterocyclylalkyl, aryl, arylalkyl, heteroaryl, and heteroarylalkyl is optionally substituted with 1-5 independently selected R²¹ substituents;
R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ᵣ-alkyl, (R¹⁸)ᵣ-cycloalkyl, (R¹⁸)ᵣ-cycloalkylalkyl, (R¹⁸)ᵣ-heterocyclyl, (R¹⁸)ᵣ-heterocyclylalkyl, (R¹⁸)ᵣ-aryl, (R¹⁸)ᵣ-arylalkyl, (R¹⁸)ᵣ-heteroaryl and (R¹⁸)ᵣ-heteroarylalkyl, wherein each r is independently 1 to 5;
Each R¹⁸ is independently selected from the group consisting of: alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
or, alternately, two R¹⁸ moieties on adjacent carbons can be linked together to form: 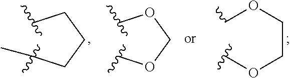
R¹⁹ is alkyl, cycloalkyl, aryl, arylalkyl or heteroarylalkyl;
R²⁰ is alkyl, cycloalkyl, aryl, halo substituted aryl, arylalkyl, heteroaryl or heteroarylalkyl;
Each R²¹ is independently selected from the group consisting of: alkyl, alkenyl, alkynyl, cycloalkyl, cycloalkenyl, heterocycloalkyl, heterocycloalkylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;
wherein each of the alkyl, cycloalkenyl, cycloalkyl, cycloalkylalkyl, heterocycloalkyl, heterocycloalkylalkyl, aryl, arylalkyl, heteroaryl, heteroarylalkyl, alkenyl and alkynyl groups in R²¹ is optionally substituted by 1 to 5 independently selected R²² groups; and
Each R²² is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

2.-13. (canceled)

14. The compound of claim 1, wherein R¹ and R³ are joined together to form a moiety selected from the group consisting of:

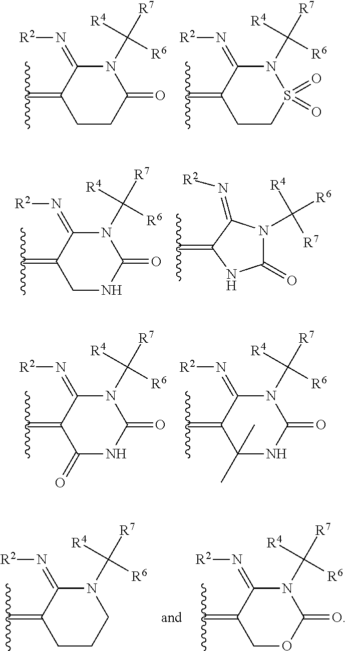

15.-22. (canceled)

23. The compound of claim 1, wherein R³ and R⁴ are joined together to form a moiety selected from the group consisting of:

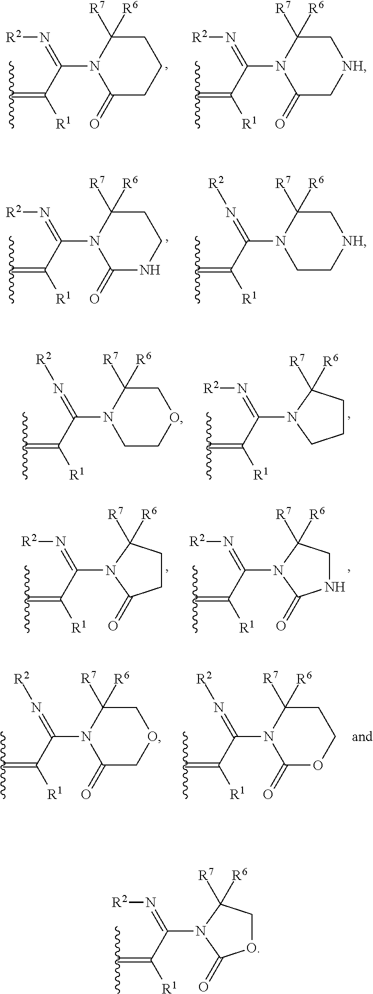

24.-33. (canceled)

35. (canceled)

36. The compound of claim 1, wherein R¹ and R³ are joined together and R³ and R⁴ are joined together to form a moiety selected from the group consisting of:

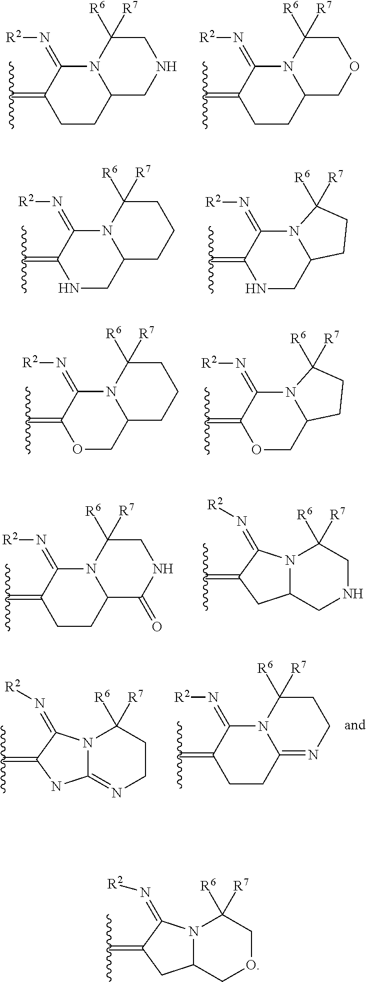

37. (canceled)

38. (canceled)

39. (canceled)

38. (canceled)

40.-48. (canceled)

49. The compound of claim 1, wherein R⁴, R⁶ and R⁷ can be the same or different, each being independently selected from the group consisting of H, alkyl and aryl-; or wherein R⁴, R⁶ and R⁷ are the same or different, each being independently selected from the group consisting of H, alkyl and aryl, wherein said aryl is substituted with 1-3 independently selected halogens.

50.-52. (canceled)

53. The compound of claim 1 wherein R² is selected from the group consisting of: H, alkyl, —OH and alkoxy.

54.-60. (canceled)

61. The compound of claim 1 wherein R¹⁰ is aryl and said aryl is unsubstituted; or wherein R¹⁰ is aryl and said aryl is substituted with 1-3 substitutents, which are the same or different, each being independently selected from the group consisting of halo, alkyl, —CN, —NH₂, —NH(alkyl), —N(alkyl)₂, hydroxy and alkoxy groups.

62.-64. (canceled)

65. The compound of claim 1 wherein R¹⁰ is unsubstituted heteroaryl; or wherein R¹⁰ is heteroaryl substituted with 1-3 substitutents, which are the same or different, each being independently selected from the group consisting of halo, alkyl, CN, NH₂, NH(alkyl), N(alkyl)₂, hydroxy and alkoxy groups.

66.-70. (canceled)

71. The compound of claim 1 wherein R⁹ is unsubstituted heteroaryl; or wherein R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups.

72.-74. (canceled)

75. The compound of claim 1 wherein:
(a) R¹ and R³ are joined together to form a 5-8 membered heterocyclyl or 5-8 membered heterocyclenyl moiety, with each of said heterocyclyl or heterocyclenyl moiety being optionally substituted with 1-5 independently selected R²¹ substituents:
(b) V is selected from the group consisting of a bond, —O—, —S(O₂)—, —S(O)—, —C(O)—, and —N(R¹⁴)—;
(c) R² is selected from the group consisting of: H, alkyl, —OH and alkoxy-;
(d) R⁴, R⁶ and R⁷ are the same or different, each being independently selected from the group consisting of H, alkyl and aryl, wherein said aryl is substituted with 1-3 independently selected halogens;
(e) R⁸ is H;
(f) R⁹ is 4-methyl-imidazol-1-yl; and
(g) R¹⁰ is

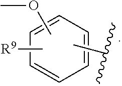

76. The compound of claim 1 wherein:
(a) R³ and R⁴ are joined together to form a 5-8 membered heterocyclyl or 5-8 membered heterocyclenyl moiety, with each of said heterocyclyl or heterocyclenyl moiety being optionally substituted with 1-5 independently selected R²¹ substituents;
(b) V is selected from the group consisting of a bond, —O—, —S(O₂)—, —S(O)—, —C(O)—, and —N(R¹⁴)—;
(c) R² is selected from the group consisting of: H, alkyl, —OH and alkoxy-;
(d) R⁶ and R⁷ are the same or different, each being independently selected from the group consisting of H, alkyl and aryl-, wherein said aryl- is substituted with 1-3 independently selected halogens;
(e) R⁸ is H;
(f) R⁹ is 4-methyl-imidazol-1-yl; and
(g) R¹⁰ is

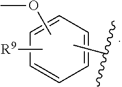

77. The compound of claim 1 wherein:
(a)
(i) R¹ and R³ are joined together to form a 5-8 membered heterocyclyl or 5-8 membered heterocyclenyl moiety, with each of said heterocyclyl or heterocyclenyl moiety being optionally substituted with 1-5 independently selected R²¹ substituents; and
(ii) R³ and R⁴ are joined together to form a 5-8 membered heterocyclyl or 5-8 membered heterocyclenyl moiety, with each of said heterocyclyl or heterocyclenyl moiety being optionally substituted with 1-5 independently selected R²¹ substituents;
(b) V is selected from the group consisting of a bond, —O—, —S(O₂)—, —S(O)—, —C(O)—, and —N(R¹⁴)—;
(c) R² is selected from the group consisting of: H, alkyl, —OH and alkoxy-;
(d) R⁶ and R⁷ are same or different, each being independently selected from the group consisting of H, alkyl and aryl-, wherein said aryl- is substituted with 1-3 independently selected halogens;
(e) R⁸ is H;
(f) R⁹ is 4-methyl-imidazol-1-yl; and
(g) R¹⁰ is

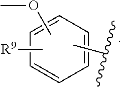

78. A compound selected from the group consisting of compounds 1 to 48 or a pharmaceutically acceptable salt thereof.

79. A compound of the formula: 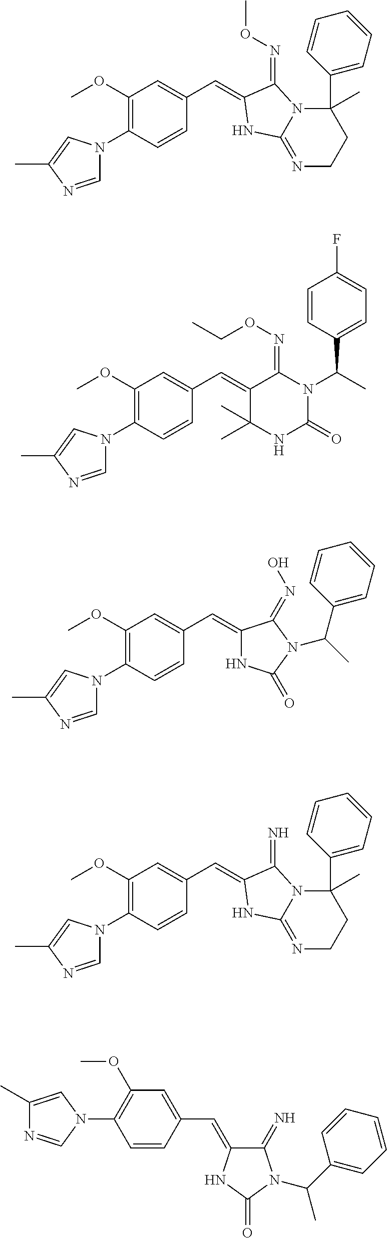 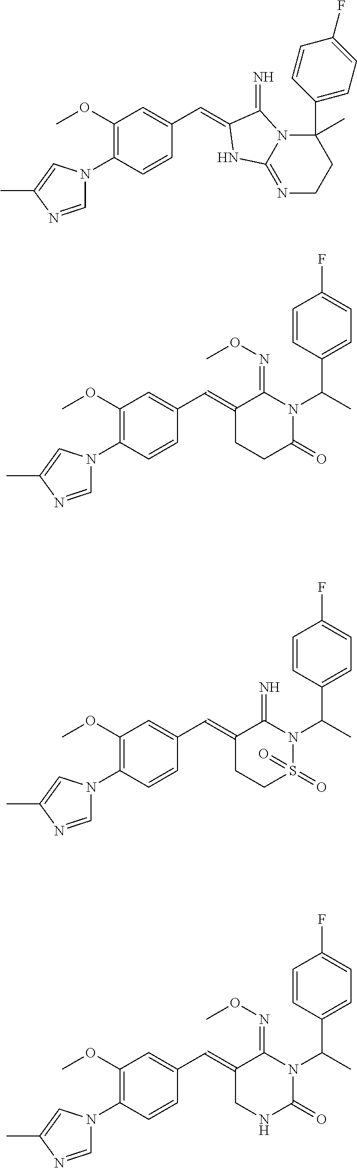 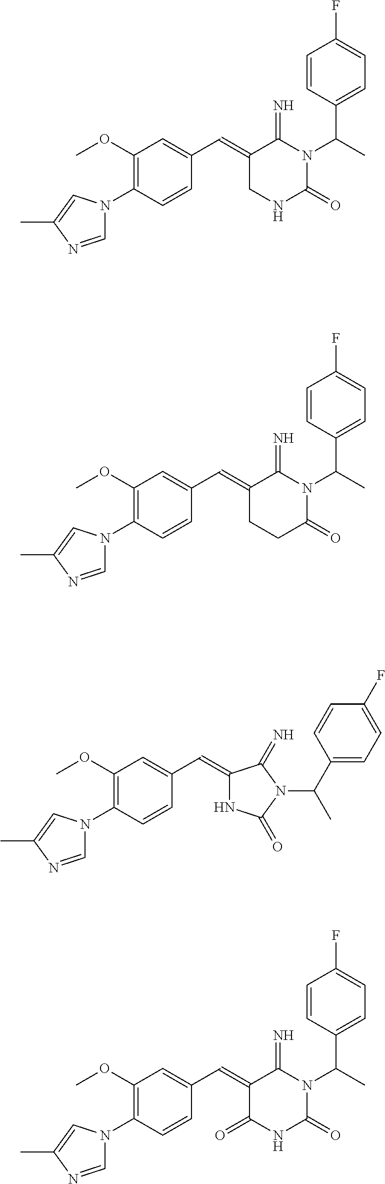 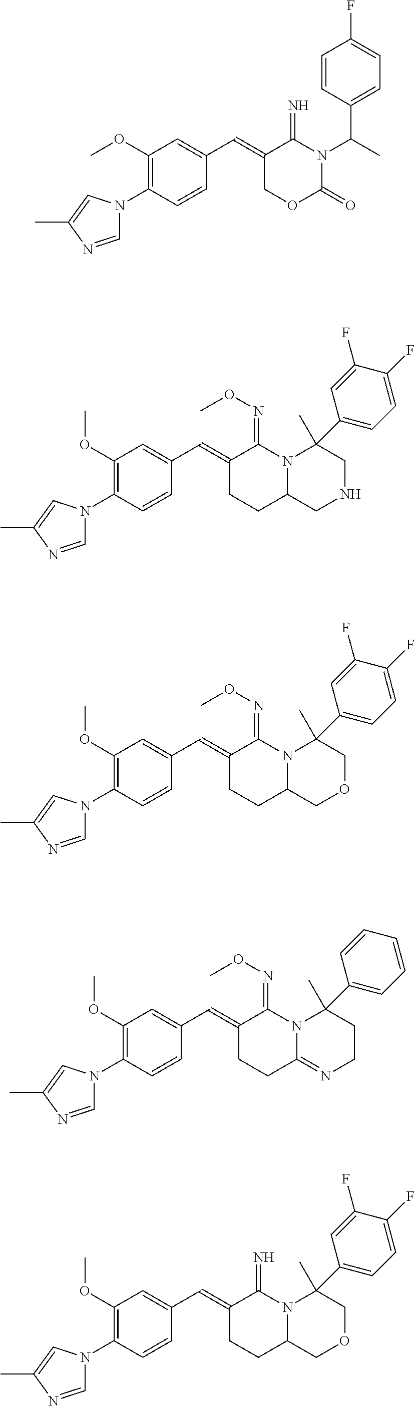 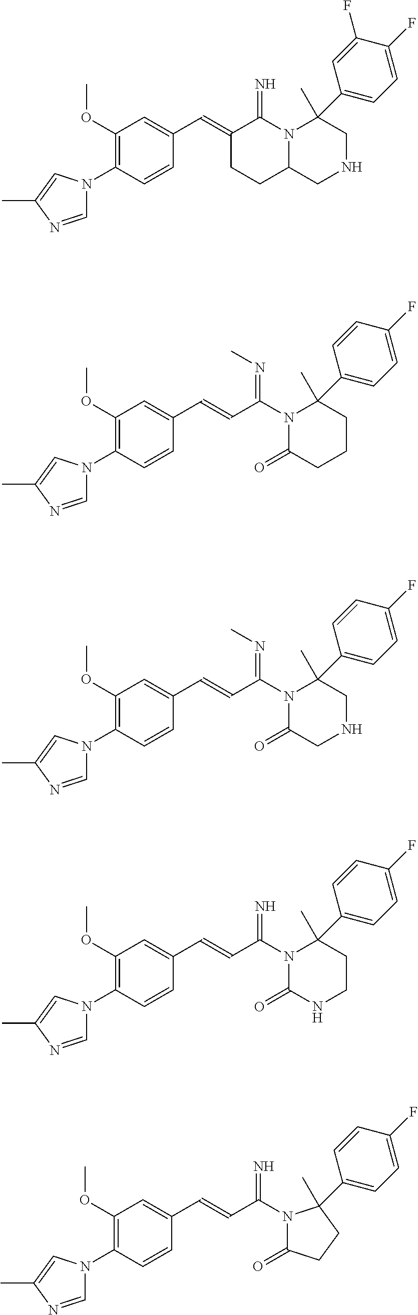 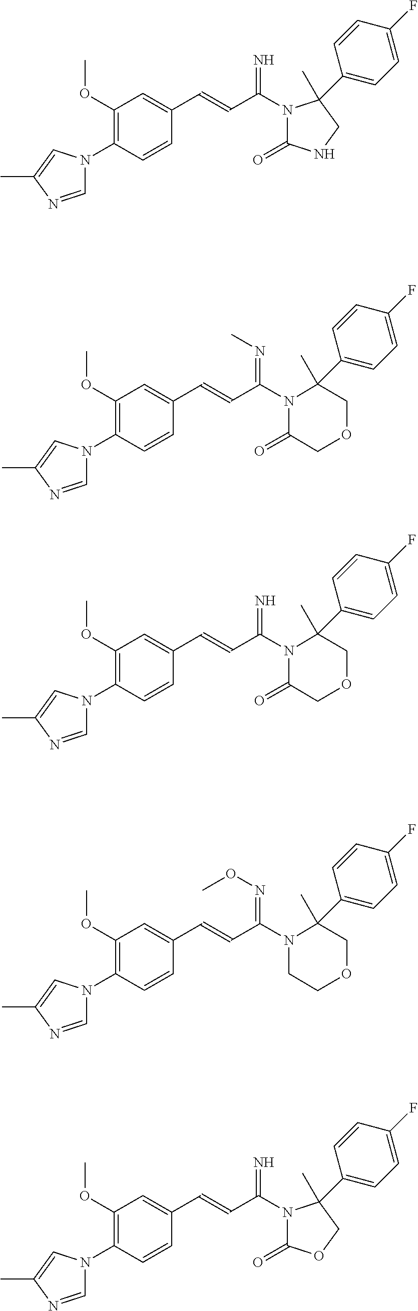
or a pharmaceutically acceptable salt thereof.

80. A composition comprising:
(1) a therapeutically effective amount of at least one compound of claim 1, or a pharmaceutically acceptable salt thereof, and at least one pharmaceutically acceptable carrier; or
(2) a therapeutically effective amount of at least one compound of claim 1, or a pharmaceutically acceptable salt thereof, and at least one pharmaceutically acceptable carrier, and a therapeutically effective amount of one or compounds selected from the group consisting of cholinesterase inhibitors, Aβ antibody inhibitors, gamma secretase inhibitors and beta secretase inhibitors; or
(3) a therapeutically effective amount of at least one compound of claim 1, or a pharmaceutically acceptable salt thereof, and at least one pharmaceutically acceptable carrier, and a therapeutically effective amount of one or compounds selected from the group consisting of cholinesterase inhibitors, Aβ antibody inhibitors, gamma secretase inhibitors and beta secretase inhibitors, wherein said cholinesterase inhibitor is donepezil hydrochloride.

81.-83. (canceled)

84. A pharmaceutical composition comprising a therapeutically effective amount of a compound of claim 1, and a pharmaceutically acceptable carrier, and an effective amount of one or more other pharmaceutically active drugs selected form the group consisting of: (a) drugs useful for the treatment of Alzheimer's disease, (b) drugs useful for inhibiting the deposition of amyloid protein (e.g., amyloid beta protein) in, on or around neurological tissue, (c) drugs useful for treating neurodegenerative diseases, and (d) drugs useful for inhibiting gamma-secretase; or a pharmaceutical composition comprising a therapeutically effective amount of a compound of claim 1, and a pharmaceutically acceptable carrier, and an effective amount of one or more BACE inhibitors.

85.-87. (canceled)

88. A pharmaceutical composition:
(1) comprising a therapeutically effective amount of at least one compound of claim 1, or a pharmaceutically acceptable salt thereof, and at least one pharmaceutically acceptable carrier, or
(2) comprising a therapeutically effective amount of at least one compound of claim 1, or a pharmaceutically acceptable salt thereof, and at least one pharmaceutically acceptable carrier, and an effective amount of one or more other pharmaceutically active drugs selected form the group consisting of: (a) drugs useful for the treatment of Alzheimer's disease, (b) drugs useful for inhibiting the deposition of amyloid protein (e.g., amyloid beta protein) in, on or around neurological tissue, (c) drugs useful for treating neurodegenerative diseases, and (d) drugs useful for inhibiting gamma-secretase, or
(3) comprising a therapeutically effective amount of at least one compound of claim 1, or a pharmaceutically acceptable salt thereof, and at least one pharmaceutically acceptable carrier, and an effective amount of one or more BACE inhibitors,
(4) comprising a therapeutically effective amount of at least one compound of claim 1, or a pharmaceutically acceptable salt thereof, and at least one pharmaceutically acceptable carrier, and effective amount of one or more cholinesterase inhibitors, or
(5) comprising a therapeutically effective amount of at least one compound of claim 1, and at least one pharmaceutically acceptable carrier, and effective amount of one or more cholinesterase inhibitors, or
(6) comprising a therapeutically effective amount of at least one compound of claim 1, or a pharmaceutically acceptable salt thereof, and at least one pharmaceutically acceptable carrier, and effective amount of one or more BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors, or
(7) comprising a therapeutically effective amount of at least one compound of claim 1, and at least one pharmaceutically acceptable carrier, and effective amount of one or more BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors, or
(8) comprising a therapeutically effective amount of at least one compound of claim 1, or a pharmaceutically acceptable salt thereof, and at least one pharmaceutically acceptable carrier, and an effective amount of donepezil hydrochloride, or
(9) comprising a therapeutically effective amount of at least one compound of claim 1, and at least one pharmaceutically acceptable carrier, and an effective amount of donepezil hydrochloride.

89. A method of:
(1) treating a central nervous system disorder comprising administering a therapeutically effective amount of at least one compound of claim 1; or
(2) treating Downs syndrome comprising administering a therapeutically effective amount of at least one compound of claim 1; or
(3) modulating gamma-secretase comprising administering an effective amount of one or more compounds of claim 1 to a patient in need of such treatment; or
(4) treating one or more neurodegenerative diseases, comprising administering an effective amount of one or more compounds of claim 1 to a patient in need of treatment; or
(5) inhibiting the deposition of amyloid protein in, on or around neurological tissue, comprising administering an effective amount of one or more compounds of claim 1 to a patient in need of treatment.

90. (canceled)

91. A method of treating Alzheimers disease comprising administering a therapeutically effective amount of at least one compound of claim 1.

92.-97. (canceled)

98. A method of (a) modulating gamma-secretase, (b) treating one or more neurodegenerative diseases, (c) inhibiting the deposition of amyloid protein in, on or around neurological tissue, or (d) treating Alzheimer's disease, comprising administering:
(1) an effective amount of a compound of claim 1, and
(2) an effective amount of one or more other pharmaceutically active ingredients selected from the group consisting of: BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors,
to a patient in need of such treatment.

99. (canceled)

100. (canceled)

101. A method of:
(1) treating Alzheimer's disease, comprising administering an effective amount of one or more compounds of claim 1, in combination with an effective amount of one or more cholinesterase, to a patient in need of treatment, or
(2) treating Alzheimer's disease, comprising administering an effective amount of one or more compounds of claim 1, in combination with an effective amount of donepezil hydrochloride, to a patient in need of treatment, or
(3) treating Alzheimer's disease, comprising administering an effective amount of a compound claim 1, in combination with an effective amount of one or more cholinesterase, to a patient in need of treatment, or
(4) treating Alzheimer's disease, comprising administering an effective amount of a compound of claim 1, in combination with an effective amount of donepezil hydrochloride, to a patient in need of treatment, or
(5) treating Alzheimer's disease, comprising administering an effective amount of one or more compounds of claim 1, in combination with an effective amount of (rivastigmine, to a patient in need of such treatment, or
(6) treating Alzheimer's disease, comprising administering an effective amount of one or more compounds of claim 1, in combination with an effective amount of tacrine, to a patient in need of such treatment, or
(7) treating Alzheimer's disease, comprising administering an effective amount of one or more compounds of claim 1, in combination with an effective amount of a Tau kinase inhibitor, to a patient in need of such treatment, or
(8) treating Alzheimer's disease, comprising administering an effective amount of one or more compounds of claim 1, in combination with an effective amount of one or more Tau kinase inhibitors selected from the group consisting of: GSK3beta inhibitors, cdk5 inhibitors, ERK inhibitors, to a patient in need of such treatment, or
(9) treating Alzheimer's disease, comprising administering an effective amount of one or more compounds of claim 1, in combination with an effective amount of one anti-Abeta vaccination, to a patient in need of such treatment, or
(10) treating Alzheimer's disease, comprising administering an effective amount of one or more compounds of claim 1, in combination with an effective amount of one or more APP ligands, to a patient in need of such treatment, or
(11) treating Alzheimer's disease, comprising administering an effective amount of one or more compounds of claim 1, in combination with an effective amount of one or more agents that upregulate insulin degrading enzyme and/or neprilysin, to a patient in need of such treatment, or
(12) treating Alzheimer's disease, comprising administering an effective amount of one or more compounds of claim 1, in combination with an effective amount of one or more cholesterol lowering agents, to a patient in need of such treatment, or
(13) treating Alzheimer's disease, comprising administering an effective amount of one or more compounds of claim 1, in combination with an effective amount of one or more cholesterol lowering agents selected from the group consisting of: Atorvastatin, Fluvastatin, Lovastatin, Mevastatin, Pitavastatin, Pravastatin, Rosuvastatin, Simvastatin, and Ezetimibe, to a patient in need of such treatment, or
(14) treating Alzheimer's disease, comprising administering an effective amount of one or more compounds of claim 1, in combination with an effective amount of one or more fibrates, to a patient in need of such treatment, or
(15) treating Alzheimer's disease, comprising administering an effective amount of one or more compounds of claim 1, in combination with an effective amount of one or more fibrates selected from the group consisting of, clofibrate, Clofibride, Etofibrate, Aluminium Clofibrate, to a patient in need of such treatment, or
(16) treating Alzheimer's disease, comprising administering an effective amount of one or more compounds of claim 1, in combination with an effective amount of one or more LXR agonists, to a patient in need of such treatment, or
(17) treating Alzheimer's disease, comprising administering an effective amount of one or more compounds of claim 1, in combination with an effective amount of one or more LRP mimics, to a patient in need of such treatment, or
(18) treating Alzheimer's disease, comprising administering an effective amount of one or more compounds of claim 1, in combination with an effective amount of one or more 5-HT6 receptor antagonists, to a patient in need of such treatment, or
(19) treating Alzheimer's disease, comprising administering an effective amount of one or more compounds of claim 1, in combination with an effective amount of one or more nicotinic receptor agonists, to a patient in need of such treatment, or
(20) treating Alzheimer's disease, comprising administering an effective amount of one or more compounds of claim 1, in combination with an effective amount of one or more H3 receptor antagonists, to a patient in need of such treatment, or
(21) treating Alzheimer's disease, comprising administering an effective amount of one or more compounds of claim 1, in combination with an effective amount of one or more histone deacetylase inhibitors, to a patient in need of such treatment, or
(22) treating Alzheimer's disease, comprising administering an effective amount of one or more compounds of claim 1, in combination with an effective amount of one or more hsp90 inhibitors, to a patient in need of such treatment, or
(23) treating Alzheimer's disease, comprising administering an effective amount of one or more compounds of claim 1, in combination with an effective amount of one or more m1 muscarinic receptor agonists, to a patient in need of such treatment, or
(24) treating Alzheimer's disease, comprising administering an effective amount of one or more compounds of claim 1, in combination with an effective amount of one or more 5-HT6 receptor antagonists mGluR1 or mGluR5 positive allosteric modulators or agonists, to a patient in need of such treatment, or
(25) treating Alzheimer's disease, comprising administering an effective amount of one or more compounds of claim 1, in combination with an effective amount of one or more mGluR2/3 antagonists, to a patient in need of such treatment, or
(26) treating Alzheimer's disease, comprising administering an effective amount of one or more compounds of formula I, in combination with an effective amount of one or more anti-inflammatory agents that can reduce neuroinflammation, to a patient in need of such treatment, or
(27) treating Alzheimer's disease, comprising administering an effective amount of one or more compounds of claim 1, in combination with an effective amount of one or more Prostaglandin EP2 receptor antagonists, to a patient in need of such treatment, or
(28) treating Alzheimer's disease, comprising administering an effective amount of one or more compounds of claim 1, in combination with an effective amount of one or more PAI-1 inhibitors, to a patient in need of such treatment, or
(29) treating Alzheimer's disease, comprising administering an effective amount of one or more compounds of claim 1, in combination with an effective amount of one or more agents that can induce Abeta efflux, to a patient in need of such treatment, or
(30) treating Alzheimer's disease, comprising administering an effective amount of one or more compounds of claim 1, in combination with an effective amount of gelsolin, to a patient in need of such treatment, or
(31) treating Downs syndrome, comprising administering an effective amount of one or more compounds of claim 1 to a patient in need of treatment, or
(32) treating Downs syndrome, comprising administering an effective amount of a compound of claim 1 to a patient in need of treatment, or
(33) treating Downs syndrome, comprising administering an effective amount of one or more compounds of claim 1, in combination with an effective amount of one or more cholinesterase inhibitors, to a patient in need of treatment.
(34) treating Downs syndrome, comprising administering an effective amount of one or more compounds of claim 1, in combination with an effective amount of donepezil hydrochloride, to a patient in need of treatment, or
(35) treating Downs syndrome, comprising administering an effective amount of a compound of claim 1, in combination with an effective amount of one or more cholinesterase inhibitors, to a patient in need of treatment.
(36) treating Downs syndrome, comprising administering an effective amount of a compound of claim 1, in combination with an effective amount of donepezil hydrochloride, to a patient in need of treatment, or
(37) treating mild cognitive impairment, comprising administering an effective amount of one or more compounds of claim 1 to a patient in need of treatment, or
(38) treating glaucoma, comprising administering an effective amount of one or more compounds of claim 1 to a patient in need of treatment, or
(39) treating cerebral amyloid angiopathy, comprising administering an effective amount of one or more compounds of claim 1 to a patient in need of treatment, or
(40) treating stroke, comprising administering an effective amount of one or more compounds of claim 1 to a patient in need of treatment, or
(41) This invention also provides a method of treating dementia, comprising administering an effective amount of one or more compounds of claim 1 to a patient in need of treatment, or
(42) treating microgliosis, comprising administering an effective amount of one or more compounds of claim 1 to a patient in need of treatment, or
(43) treating brain inflammation, comprising administering an effective amount of one or more compounds of claim 1 to a patient in need of treatment, or
(44) treating olfactory function loss comprising administering an effective amount of one or more compounds of claim 1 to a patient in need of treatment, or
(45) treating Alzheimer's disease, comprising administering an effective amount of a compound of claim 1, and an effective amount of one or more compounds selected from the group consisting of Aβ antibody inhibitors, gamma secretase inhibitors and beta secretase inhibitors, to a patient in need of such treatment, or
(46) treating Alzheimer's disease, comprising administering an effective amount of a compound of claim 1, and an effective amount of one or more BACE inhibitors, to a patient in need of such treatment.

102. A kit comprising, in separate containers, in a single package, pharmaceutical compositions for use in combination, wherein one container comprises an effective amount of a compound of claim 1 in a pharmaceutically acceptable carrier, and another container comprises an effective amount of another pharmaceutically active ingredient, the combined quantities of the compound of claim 1 and the other pharmaceutically active ingredient being effective to: (a) treat Alzheimer's disease, or (b) inhibit the deposition of amyloid protein in, on or around neurological tissue, or (c) treat neurodegenerative diseases, or (d) modulate the activity of gamma-secretase.

103.-110. (canceled)
